SEQUENCE LISTING (592 sequences)

SEQ ID NO: 1 - DNA, 20 bp, Artificial Sequence
  Description of Artificial Sequence Synthetic oligonucleotide
aaagccgctcaactacatgg

SEQ ID NO: 2 - DNA, 23 bp, Artificial Sequence
  Description of Artificial Sequence Synthetic oligonucleotide
ctctatttccttcagttctgctc

SEQ ID NO: 3 - DNA, 21 bp, Artificial Sequence
  Description of Artificial Sequence Synthetic oligonucleotide
tgactacttttgacttcagcc

SEQ ID NO: 4 - DNA, 22 bp, Artificial Sequence
  Description of Artificial Sequence Synthetic oligonucleotide
aaccattcaacatttttaaccc

SEQ ID NO: 5 - DNA, 47 bp, Artificial Sequence
  Description of Artificial Sequence Synthetic oligonucleotide
gcctccctcgcgccatcagacgctttttgtgtgtgttttctcttcca

SEQ ID NO: 6 - DNA, 50 bp, Artificial Sequence
  Description of Artificial Sequence Synthetic oligonucleotide
gccttgccagcccgctcagacgcttcataatctgtgtattcaaacagtgc

SEQ ID NO: 7 - DNA, 51 bp, Artificial Sequence
  Description of Artificial Sequence Synthetic oligonucleotide
gcctccctcgcgccatcagacgctgccgtatgacataattactattctttt

SEQ ID NO: 8 - DNA, 44 bp, Artificial Sequence
  Description of Artificial Sequence Synthetic oligonucleotide
gccttgccagcccgctcagacgctcccactcaaattcaggcaat

SEQ ID NO: 9 - DNA, 20 bp, Artificial Sequence
  Description of Artificial Sequence Synthetic oligonucleotide
gctctccgccttttctctct

SEQ ID NO: 10 - DNA, 20 bp, Artificial Sequence
  Description of Artificial Sequence Synthetic oligonucleotide
gagcggagccaaataatcac

SEQ ID NO: 11 - DNA, 20 bp, Artificial Sequence
  Description of Artificial Sequence Synthetic oligonucleotide
cgatgctatgagagggaagc

SEQ ID NO: 12 - DNA, 20 bp, Artificial Sequence
  Description of Artificial Sequence Synthetic oligonucleotide
ccaggagcaagtccgtctta

SEQ ID NO: 13 - DNA, 23 bp, Artificial Sequence
  Description of Artificial Sequence Synthetic oligonucleotide
ttttgtgtgtgttttctcttcca

SEQ ID NO: 14 - DNA, 26 bp, Artificial Sequence
  Description of Artificial Sequence Synthetic oligonucleotide
tcataatctgtgtattcaaacagtgc

SEQ ID NO: 15 - DNA, 24 bp, Artificial Sequence
  Description of Artificial Sequence Synthetic oligonucleotide
aacaaccaaagctatgtgtatgga

SEQ ID NO: 16 - DNA, 20 bp, Artificial Sequence
  Description of Artificial Sequence Synthetic oligonucleotide
catgtgcaaaacacaaccaa

SEQ ID NO: 17 - DNA, 24 bp, Artificial Sequence
  Description of Artificial Sequence Synthetic oligonucleotide
tttctcctttcttttctttttcca

SEQ ID NO: 18 - DNA, 20 bp, Artificial Sequence
  Description of Artificial Sequence Synthetic oligonucleotide
agcatctccaaaaccattgc

SEQ ID NO: 19 - DNA, 21 bp, Artificial Sequence
  Description of Artificial Sequence Synthetic oligonucleotide
ccatcctgaaatgtgatgctt

SEQ ID NO: 20 - DNA, 20 bp, Artificial Sequence
  Description of Artificial Sequence Synthetic oligonucleotide
tcctttgtggcatctcttcc

SEQ ID NO: 21 - DNA, 21 bp, Artificial Sequence
  Description of Artificial Sequence Synthetic oligonucleotide
ggacatttgctctgttggttt

SEQ ID NO: 22 - DNA, 23 bp, Artificial Sequence
  Description of Artificial Sequence Synthetic oligonucleotide
ttcatggctagaaaggatcaaaa

SEQ ID NO: 23 - DNA, 20 bp, Artificial Sequence
  Description of Artificial Sequence Synthetic oligonucleotide
gactggaagtgctgtgtgga

SEQ ID NO: 24 - DNA, 20 bp, Artificial Sequence
  Description of Artificial Sequence Synthetic oligonucleotide
tgggatcgcttcttaactgc

SEQ ID NO: 25 - DNA, 22 bp, Artificial Sequence
  Description of Artificial Sequence Synthetic oligonucleotide
cagcattttgacaatgtttctg

SEQ ID NO: 26 - DNA, 25 bp, Artificial Sequence
  Description of Artificial Sequence Synthetic oligonucleotide
tctttacctcaaaagaacaaaagag

SEQ ID NO: 27 - DNA, 20 bp, Artificial Sequence
  Description of Artificial Sequence Synthetic oligonucleotide
ccagggaagctacctggaat

SEQ ID NO: 28 - DNA, 24 bp, Artificial Sequence
  Description of Artificial Sequence Synthetic oligonucleotide
ttttaagccaacaaatttgacaac

SEQ ID NO: 29 - DNA, 20 bp, Artificial Sequence
  Description of Artificial Sequence Synthetic oligonucleotide
agcagccattttgctgattt

SEQ ID NO: 30 - DNA, 23 bp, Artificial Sequence
  Description of Artificial Sequence Synthetic oligonucleotide
aagagataatccaggctttaccc

SEQ ID NO: 31 - DNA, 22 bp, Artificial Sequence
  Description of Artificial Sequence Synthetic oligonucleotide
aacgtcgtttctatgtgtttcc

SEQ ID NO: 32 - DNA, 20 bp, Artificial Sequence
  Description of Artificial Sequence Synthetic oligonucleotide
gggctgtttgcttcaacaat

SEQ ID NO: 33 - DNA, 23 bp, Artificial Sequence
  Description of Artificial Sequence Synthetic oligonucleotide
tcatgggaagtacctgttgttct

SEQ ID NO: 34 - DNA, 20 bp, Artificial Sequence
  Description of Artificial Sequence Synthetic oligonucleotide
cgcactgcatgcataagaga

SEQ ID NO: 35 - DNA, 20 bp, Artificial Sequence
  Description of Artificial Sequence Synthetic oligonucleotide
cagcaagaccctcctttcag

SEQ ID NO: 36 - DNA, 20 bp, Artificial Sequence
  Description of Artificial Sequence Synthetic oligonucleotide
ctctgccctctcttggaatg

SEQ ID NO: 37 - DNA, 21 bp, Artificial Sequence
  Description of Artificial Sequence Synthetic oligonucleotide
aagggttcatttgtttttcca

SEQ ID NO: 38 - DNA, 20 bp, Artificial Sequence
  Description of Artificial Sequence Synthetic oligonucleotide
ggagtccaggtcaaggcata

SEQ ID NO: 39 - DNA, 24 bp, Artificial Sequence
  Description of Artificial Sequence Synthetic oligonucleotide
ttggatatgtgcataactgtttga

SEQ ID NO: 40 - DNA, 20 bp, Artificial Sequence
  Description of Artificial Sequence Synthetic oligonucleotide
gtgttgactgtgtgggcgta

SEQ ID NO: 41 - DNA, 24 bp, Artificial Sequence
  Description of Artificial Sequence Synthetic oligonucleotide
tgaatgctttataagcttgttctg

SEQ ID NO: 42 - DNA, 20 bp, Artificial Sequence
  Description of Artificial Sequence Synthetic oligonucleotide
ggtttgtgtgtgtgcctgaa

SEQ ID NO: 43 - DNA, 20 bp, Artificial Sequence
  Description of Artificial Sequence Synthetic oligonucleotide
tggcttttatccgggtattg

SEQ ID NO: 44 - DNA, 21 bp, Artificial Sequence
  Description of Artificial Sequence Synthetic oligonucleotide
ttgggtccaaatgaagactaa

SEQ ID NO: 45 - DNA, 21 bp, Artificial Sequence
  Description of Artificial Sequence Synthetic oligonucleotide
tctgcatgtcttttctgatgg

SEQ ID NO: 46 - DNA, 19 bp, Artificial Sequence
  Description of Artificial Sequence Synthetic oligonucleotide
ctgtcaagggaaggtgcaa

SEQ ID NO: 47 - DNA, 20 bp, Artificial Sequence
  Description of Artificial Sequence Synthetic oligonucleotide
ctgcattcgttctgtgcatc

SEQ ID NO: 48 - DNA, 20 bp, Artificial Sequence
  Description of Artificial Sequence Synthetic oligonucleotide
ttgcctcactgagctgtacc

SEQ ID NO: 49 - DNA, 21 bp, Artificial Sequence
  Description of Artificial Sequence Synthetic oligonucleotide
tgttctggcaatcattgtgaa

SEQ ID NO: 50 - DNA, 21 bp, Artificial Sequence
  Description of Artificial Sequence Synthetic oligonucleotide
tgttttgttcccgtttttctg

SEQ ID NO: 51 - DNA, 20 bp, Artificial Sequence
  Description of Artificial Sequence Synthetic oligonucleotide
ccccgtgtgtctctctctgt

SEQ ID NO: 52 - DNA, 22 bp, Artificial Sequence
  Description of Artificial Sequence Synthetic oligonucleotide
gagaaacagcaaacaagaccaa

SEQ ID NO: 53 - DNA, 20 bp, Artificial Sequence
  Description of Artificial Sequence Synthetic oligonucleotide
cccaaatcctcatgcattta

SEQ ID NO: 54 - DNA, 20 bp, Artificial Sequence
  Description of Artificial Sequence Synthetic oligonucleotide
aaagcgtgtgaccacctacc

SEQ ID NO: 55 - DNA, 23 bp, Artificial Sequence
  Description of Artificial Sequence Synthetic oligonucleotide
tgaatcctctttctctcatgtcc

SEQ ID NO: 56 - DNA, 21 bp, Artificial Sequence
  Description of Artificial Sequence Synthetic oligonucleotide
tggagtcatcatgttggagaa

SEQ ID NO: 57 - DNA, 20 bp, Artificial Sequence
  Description of Artificial Sequence Synthetic oligonucleotide
gcatagagggactcatgctg

SEQ ID NO: 58 - DNA, 25 bp, Artificial Sequence
  Description of Artificial Sequence Synthetic oligonucleotide
tcagatatgtcctgacaaaatgaaa

SEQ ID NO: 59 - DNA, 20 bp, Artificial Sequence
  Description of Artificial Sequence Synthetic oligonucleotide
tccttgcctttctctcttgc

SEQ ID NO: 60 - DNA, 21 bp, Artificial Sequence
  Description of Artificial Sequence Synthetic oligonucleotide
tttgcacaatggtatgactgc

SEQ ID NO: 61 - DNA, 20 bp, Artificial Sequence
  Description of Artificial Sequence Synthetic oligonucleotide
tgtgatgcctgaagctgaac

SEQ ID NO: 62 - DNA, 24 bp, Artificial Sequence
  Description of Artificial Sequence Synthetic oligonucleotide
ttttctcaaatcactgaaaccaaa

SEQ ID NO: 63 - DNA, 22 bp, Artificial Sequence
  Description of Artificial Sequence Synthetic oligonucleotide
agcgctactgtgttctctcttg

SEQ ID NO: 64 - DNA, 19 bp, Artificial Sequence
  Description of Artificial Sequence Synthetic oligonucleotide
gcacgctgtgccacctact

SEQ ID NO: 65 - DNA, 20 bp, Artificial Sequence
  Description of Artificial Sequence Synthetic oligonucleotide
atgttttccagcctgtgacc

SEQ ID NO: 66 - DNA, 20 bp, Artificial Sequence
  Description of Artificial Sequence Synthetic oligonucleotide
caacatgcatccattcctga

SEQ ID NO: 67 - DNA, 20 bp, Artificial Sequence
  Description of Artificial Sequence Synthetic oligonucleotide
gcgttcttcgctgtccttac

SEQ ID NO: 68 - DNA, 20 bp, Artificial Sequence
  Description of Artificial Sequence Synthetic oligonucleotide
atgacaaagtccgggttcac

SEQ ID NO: 69 - DNA, 27 bp, Artificial Sequence
  Description of Artificial Sequence Synthetic oligonucleotide
gccgtatgacataattactattctttt

SEQ ID NO: 70 - DNA, 20 bp, Artificial Sequence
  Description of Artificial Sequence Synthetic oligonucleotide
cccactcaaattcaggcaat

SEQ ID NO: 71 - DNA, 20 bp, Artificial Sequence
  Description of Artificial Sequence Synthetic oligonucleotide
atccctgctctcatgcagtt

SEQ ID NO: 72 - DNA, 22 bp, Artificial Sequence
  Description of Artificial Sequence Synthetic oligonucleotide
tgagatgctgcattttaaagga

SEQ ID NO: 73 - DNA, 20 bp, Artificial Sequence
  Description of Artificial Sequence Synthetic oligonucleotide
gctttggctcacctatcacc

SEQ ID NO: 74 - DNA, 25 bp, Artificial Sequence
  Description of Artificial Sequence Synthetic oligonucleotide
cactgaaaggaaatatgacaatgaa

SEQ ID NO: 75 - DNA, 20 bp, Artificial Sequence
  Description of Artificial Sequence Synthetic oligonucleotide
gccaacatcccacctcttat

SEQ ID NO: 76 - DNA, 20 bp, Artificial Sequence
  Description of Artificial Sequence Synthetic oligonucleotide
tccccctagctgtctgaact

SEQ ID NO: 77 - DNA, 20 bp, Artificial Sequence
  Description of Artificial Sequence Synthetic oligonucleotide
acggtgctgtgtgttttctg

SEQ ID NO: 78 - DNA, 20 bp, Artificial Sequence
  Description of Artificial Sequence Synthetic oligonucleotide
tggagacatgtggagtgacg

SEQ ID NO: 79 - DNA, 21 bp, Artificial Sequence
  Description of Artificial Sequence Synthetic oligonucleotide
cggctgtgttctgtactttgc

SEQ ID NO: 80 - DNA, 19 bp, Artificial Sequence
  Description of Artificial Sequence Synthetic oligonucleotide
actctttggcaccaccaca

SEQ ID NO: 81 - DNA, 20 bp, Artificial Sequence
  Description of Artificial Sequence Synthetic oligonucleotide
atcaacactgtcccacagca

SEQ ID NO: 82 - DNA, 20 bp, Artificial Sequence
  Description of Artificial Sequence Synthetic oligonucleotide
cagctgagccctgttgtaga

SEQ ID NO: 83 - DNA, 20 bp, Artificial Sequence
  Description of Artificial Sequence Synthetic oligonucleotide
tcatgaaaaaccccaaaacc

SEQ ID NO: 84 - DNA, 21 bp, Artificial Sequence
  Description of Artificial Sequence Synthetic oligonucleotide
tgagtcacacaggtgaggaaa

SEQ ID NO: 85 - DNA, 27 bp, Artificial Sequence
  Description of Artificial Sequence Synthetic oligonucleotide
tgttacaattctatctgcattgtttct

SEQ ID NO: 86 - DNA, 25 bp, Artificial Sequence
  Description of Artificial Sequence Synthetic oligonucleotide
tgactcagtataacagtggaagctg

SEQ ID NO: 87 - DNA, 24 bp, Artificial Sequence
  Description of Artificial Sequence Synthetic oligonucleotide
ttctagcatttgtctttgcatttc

SEQ ID NO: 88 - DNA, 20 bp, Artificial Sequence
  Description of Artificial Sequence Synthetic oligonucleotide
gagctgcacaaagatgacca

SEQ ID NO: 89 - DNA, 26 bp, Artificial Sequence
  Description of Artificial Sequence Synthetic oligonucleotide
tgcagatattctcaaacatttaatcc

SEQ ID NO: 90 - DNA, 20 bp, Artificial Sequence
  Description of Artificial Sequence Synthetic oligonucleotide
gcattctttgaccccaagag

SEQ ID NO: 91 - DNA, 20 bp, Artificial Sequence
  Description of Artificial Sequence Synthetic oligonucleotide
aggacctgcaggactgacat

SEQ ID NO: 92 - DNA, 21 bp, Artificial Sequence
  Description of Artificial Sequence Synthetic oligonucleotide
tgccaacagaataaggcattt

SEQ ID NO: 93 - DNA, 23 bp, Artificial Sequence
  Description of Artificial Sequence Synthetic oligonucleotide
cattaagtttgcatgtttttcca

SEQ ID NO: 94 - DNA, 20 bp, Artificial Sequence
  Description of Artificial Sequence Synthetic oligonucleotide
ggtttcggttctcgttgttg

SEQ ID NO: 95 - DNA, 25 bp, Artificial Sequence
  Description of Artificial Sequence Synthetic oligonucleotide
gctttattttgtctgtttgtttctt

SEQ ID NO: 96 - DNA, 20 bp, Artificial Sequence
  Description of Artificial Sequence Synthetic oligonucleotide
ccccatgcaaagtgatgaac

SEQ ID NO: 97 - DNA, 20 bp, Artificial Sequence
  Description of Artificial Sequence Synthetic oligonucleotide
agctgtatggcgcttgtttt

SEQ ID NO: 98 - DNA, 25 bp, Artificial Sequence
  Description of Artificial Sequence Synthetic oligonucleotide
cagatattttgacttgagaggttct

SEQ ID NO: 99 - DNA, 20 bp, Artificial Sequence
  Description of Artificial Sequence Synthetic oligonucleotide
atccttagccagccacctct

SEQ ID NO: 100 - DNA, 20 bp, Artificial Sequence
  Description of Artificial Sequence Synthetic oligonucleotide
acactgcaggaaacaccaca

SEQ ID NO: 101 - DNA, 22 bp, Artificial Sequence
  Description of Artificial Sequence Synthetic oligonucleotide
caactgcaagatgaatgttttg

SEQ ID NO: 102 - DNA, 20 bp, Artificial Sequence
  Description of Artificial Sequence Synthetic oligonucleotide
tgtcgtgctgaattcatggt

SEQ ID NO: 103 - DNA, 20 bp, Artificial Sequence
  Description of Artificial Sequence Synthetic oligonucleotide
tcctggtttgttgcattgac

SEQ ID NO: 104 - DNA, 24 bp, Artificial Sequence
  Description of Artificial Sequence Synthetic oligonucleotide
caaagaaatgcagaccaatacaaa

SEQ ID NO: 105 - DNA, 23 bp, Artificial Sequence
  Description of Artificial Sequence Synthetic oligonucleotide
gcaaacttttgctggtacttttc

SEQ ID NO: 106 - DNA, 27 bp, Artificial Sequence
  Description of Artificial Sequence Synthetic oligonucleotide
tcactgctctacaaagattaaagaaac

SEQ ID NO: 107 - DNA, 20 bp, Artificial Sequence
  Description of Artificial Sequence Synthetic oligonucleotide
atgcatttggtaggtgctca

SEQ ID NO: 108 - DNA, 27 bp, Artificial Sequence
  Description of Artificial Sequence Synthetic oligonucleotide
attaagcaacaaatttcctaatgtagt

SEQ ID NO: 109 - DNA, 21 bp, Artificial Sequence
  Description of Artificial Sequence Synthetic oligonucleotide
tttctgtgtgtgtctgtctgc

SEQ ID NO: 110 - DNA, 20 bp, Artificial Sequence
  Description of Artificial Sequence Synthetic oligonucleotide
acaaggctggggatgaaaac

SEQ ID NO: 111 - DNA, 25 bp, Artificial Sequence
  Description of Artificial Sequence Synthetic oligonucleotide
gcctacatttgtttcatttcttttc

SEQ ID NO: 112 - DNA, 19 bp, Artificial Sequence
  Description of Artificial Sequence Synthetic oligonucleotide
ccacaaagatgggcagatg

SEQ ID NO: 113 - DNA, 20 bp, Artificial Sequence
  Description of Artificial Sequence Synthetic oligonucleotide
agcacggtctgatgttttcc

SEQ ID NO: 114 - DNA, 21 bp, Artificial Sequence
  Description of Artificial Sequence Synthetic oligonucleotide
ctaggatgccgtgactttcag

SEQ ID NO: 115 - DNA, 25 bp, Artificial Sequence
  Description of Artificial Sequence Synthetic oligonucleotide
ttgatgttcattttattgtttttcc

SEQ ID NO: 116 - DNA, 21 bp, Artificial Sequence
  Description of Artificial Sequence Synthetic oligonucleotide
aagtgccagaacacagatgaa

SEQ ID NO: 117 - DNA, 27 bp, Artificial Sequence
  Description of Artificial Sequence Synthetic oligonucleotide
ttgacttgataataaaagttgtgaatg

SEQ ID NO: 118 - DNA, 20 bp, Artificial Sequence
  Description of Artificial Sequence Synthetic oligonucleotide
cgatgaattcgtccacaaag

SEQ ID NO: 119 - DNA, 22 bp, Artificial Sequence
  Description of Artificial Sequence Synthetic oligonucleotide
caatgacgttgacctgtctctc

SEQ ID NO: 120 - DNA, 20 bp, Artificial Sequence
  Description of Artificial Sequence Synthetic oligonucleotide
tctcagagcaaagggctcat

SEQ ID NO: 121 - DNA, 24 bp, Artificial Sequence
  Description of Artificial Sequence Synthetic oligonucleotide
tggtgtgtgaaatgttttattttc

SEQ ID NO: 122 - DNA, 21 bp, Artificial Sequence
  Description of Artificial Sequence Synthetic oligonucleotide
ctctgcacagaaattgggaat

SEQ ID NO: 123 - DNA, 24 bp, Artificial Sequence
  Description of Artificial Sequence Synthetic oligonucleotide
tggaaggatgtactcaattgtttt

SEQ ID NO: 124 - DNA, 24 bp, Artificial Sequence
  Description of Artificial Sequence Synthetic oligonucleotide
tctgtaattctgtcagttcactcg

SEQ ID NO: 125 - DNA, 20 bp, Artificial Sequence
  Description of Artificial Sequence Synthetic oligonucleotide
ttgtggattgccgtatgtgt

SEQ ID NO: 126 - DNA, 21 bp, Artificial Sequence
  Description of Artificial Sequence Synthetic oligonucleotide
ggaccttaaggcttctggcta

SEQ ID NO: 127 - DNA, 20 bp, Artificial Sequence
  Description of Artificial Sequence Synthetic oligonucleotide
gtggtggtgttcttgtttgc

SEQ ID NO: 128 - DNA, 23 bp, Artificial Sequence
  Description of Artificial Sequence Synthetic oligonucleotide
tctgagtctaccatgatgtctgc

SEQ ID NO: 129 - DNA, 22 bp, Artificial Sequence
  Description of Artificial Sequence Synthetic oligonucleotide
tgattctttcctgttccctgtt

SEQ ID NO: 130 - DNA, 24 bp, Artificial Sequence
  Description of Artificial Sequence Synthetic oligonucleotide
cacatctgtaaaatccaaagagtc

SEQ ID NO: 131 - DNA, 20 bp, Artificial Sequence
  Description of Artificial Sequence Synthetic oligonucleotide
gctccggaatgttgatcttg

SEQ ID NO: 132 - DNA, 20 bp, Artificial Sequence
  Description of Artificial Sequence Synthetic oligonucleotide
gcggaacaaattctgctgta

SEQ ID NO: 133 - DNA, 21 bp, Artificial Sequence
  Description of Artificial Sequence Synthetic oligonucleotide
gaaggctgacttcttcctggt

SEQ ID NO: 134 - DNA, 20 bp, Artificial Sequence
  Description of Artificial Sequence Synthetic oligonucleotide
actcccaggtatccccagag

SEQ ID NO: 135 - DNA, 20 bp, Artificial Sequence
  Description of Artificial Sequence Synthetic oligonucleotide
tggtgccctcttttcttttt

SEQ ID NO: 136 - DNA, 20 bp, Artificial Sequence
  Description of Artificial Sequence Synthetic oligonucleotide
gaaatcccctttgaacgaat

SEQ ID NO: 137 - DNA, 20 bp, Artificial Sequence
  Description of Artificial Sequence Synthetic oligonucleotide
actcgggcgtgtactttcag

SEQ ID NO: 138 - DNA, 20 bp, Artificial Sequence
  Description of Artificial Sequence Synthetic oligonucleotide
atgggggtggaagaaatcac

SEQ ID NO: 139 - DNA, 26 bp, Artificial Sequence
  Description of Artificial Sequence Synthetic oligonucleotide
caaagtctcaatgtttcctaatgttg

SEQ ID NO: 140 - DNA, 22 bp, Artificial Sequence
  Description of Artificial Sequence Synthetic oligonucleotide
ctttggaaaagtatgccactcc

SEQ ID NO: 141 - DNA, 22 bp, Artificial Sequence
  Description of Artificial Sequence Synthetic oligonucleotide
ggagaaaggaaaattgcttcaa

SEQ ID NO: 142 - DNA, 20 bp, Artificial Sequence
  Description of Artificial Sequence Synthetic oligonucleotide
cgtggactaacggattcaga

SEQ ID NO: 143 - DNA, 20 bp, Artificial Sequence
  Description of Artificial Sequence Synthetic oligonucleotide
tgtgtgtgtgctgacattgg

SEQ ID NO: 144 - DNA, 20 bp, Artificial Sequence
  Description of Artificial Sequence Synthetic oligonucleotide
ctgctcacaccatgcgttta

SEQ ID NO: 145 - DNA, 20 bp, Artificial Sequence
  Description of Artificial Sequence Synthetic oligonucleotide
tctccgtaccacattccaca

SEQ ID NO: 146 - DNA, 21 bp, Artificial Sequence
  Description of Artificial Sequence Synthetic oligonucleotide
cctgtgctttgtcacagacct

SEQ ID NO: 147 - DNA, 23 bp, Artificial Sequence
  Description of Artificial Sequence Synthetic oligonucleotide
ttcatcccttcgtataccttttt

SEQ ID NO: 148 - DNA, 20 bp, Artificial Sequence
  Description of Artificial Sequence Synthetic oligonucleotide
tcattggaaagccaaactga

SEQ ID NO: 149 - DNA, 27 bp, Artificial Sequence
  Description of Artificial Sequence Synthetic oligonucleotide
ttcgtatccatttacaaaatttatcat

SEQ ID NO: 150 - DNA, 22 bp, Artificial Sequence
  Description of Artificial Sequence Synthetic oligonucleotide
ctcaaacagatggtgtttctgc

SEQ ID NO: 151 - DNA, 20 bp, Artificial Sequence
  Description of Artificial Sequence Synthetic oligonucleotide
gtcccagctgctaaatctgc

SEQ ID NO: 152 - DNA, 20 bp, Artificial Sequence
  Description of Artificial Sequence Synthetic oligonucleotide
acatatccacgagcccagac

SEQ ID NO: 153 - DNA, 21 bp, Artificial Sequence
  Description of Artificial Sequence Synthetic oligonucleotide
cacgtcatactcaatgcctca

SEQ ID NO: 154 - DNA, 20 bp, Artificial Sequence
  Description of Artificial Sequence Synthetic oligonucleotide
tgggggaatgtttagcttga

SEQ ID NO: 155 - DNA, 44 bp, Artificial Sequence
  Description of Artificial Sequence Synthetic oligonucleotide
gcctccctcgcgccatcagacgctgctctccgccttttctctct

SEQ ID NO: 156 - DNA, 44 bp, Artificial Sequence
  Description of Artificial Sequence Synthetic oligonucleotide
gccttgccagcccgctcagacgctgagcggagccaaataatcac

SEQ ID NO: 157 - DNA, 44 bp, Artificial Sequence
  Description of Artificial Sequence Synthetic oligonucleotide
gcctccctcgcgccatcagacgctcgatgctatgagagggaagc

SEQ ID NO: 158 - DNA, 44 bp, Artificial Sequence
  Description of Artificial Sequence Synthetic oligonucleotide
gccttgccagcccgctcagacgctccaggagcaagtccgtctta

SEQ ID NO: 159 - DNA, 47 bp, Artificial Sequence; identical to SEQ ID NO: 5
  Description of Artificial Sequence Synthetic oligonucleotide

SEQ ID NO: 160 - DNA, 50 bp, Artificial Sequence; identical to SEQ ID NO: 6
  Description of Artificial Sequence Synthetic oligonucleotide

SEQ ID NO: 161 - DNA, 48 bp, Artificial Sequence
  Description of Artificial Sequence Synthetic oligonucleotide
gcctccctcgcgccatcagacgctaacaaccaaagctatgtgtatgga

SEQ ID NO: 162 - DNA, 44 bp, Artificial Sequence
  Description of Artificial Sequence Synthetic oligonucleotide
gccttgccagcccgctcagacgctcatgtgcaaaacacaaccaa

SEQ ID NO: 163 - DNA, 48 bp, Artificial Sequence
  Description of Artificial Sequence Synthetic oligonucleotide
gcctccctcgcgccatcagacgcttttctcctttcttttctttttcca

SEQ ID NO: 164 - DNA, 44 bp, Artificial Sequence
  Description of Artificial Sequence Synthetic oligonucleotide
gccttgccagcccgctcagacgctagcatctccaaaaccattgc

SEQ ID NO: 165 - DNA, 45 bp, Artificial Sequence
  Description of Artificial Sequence Synthetic oligonucleotide
gcctccctcgcgccatcagacgctccatcctgaaatgtgatgctt

SEQ ID NO: 166 - DNA, 44 bp, Artificial Sequence
  Description of Artificial Sequence Synthetic oligonucleotide
gccttgccagcccgctcagacgcttcctttgtggcatctcttcc

SEQ ID NO: 167 - DNA, 45 bp, Artificial Sequence
  Description of Artificial Sequence Synthetic oligonucleotide
gcctccctcgcgccatcagacgctggacatttgctctgttggttt

SEQ ID NO: 168 - DNA, 47 bp, Artificial Sequence
  Description of Artificial Sequence Synthetic oligonucleotide
gccttgccagcccgctcagacgctttcatggctagaaaggatcaaaa

SEQ ID NO: 169 - DNA, 44 bp, Artificial Sequence
  Description of Artificial Sequence Synthetic oligonucleotide
gcctccctcgcgccatcagacgctgactggaagtgctgtgtgga

SEQ ID NO: 170 - DNA, 44 bp, Artificial Sequence
  Description of Artificial Sequence Synthetic oligonucleotide
gccttgccagcccgctcagacgcttgggatcgcttcttaactgc

SEQ ID NO: 171 - DNA, 46 bp, Artificial Sequence
  Description of Artificial Sequence Synthetic oligonucleotide
gcctccctcgcgccatcagacgctcagcattttgacaatgtttctg

SEQ ID NO: 172 - DNA, 49 bp, Artificial Sequence
  Description of Artificial Sequence Synthetic oligonucleotide
gccttgccagcccgctcagacgcttctttacctcaaaagaacaaaagag

SEQ ID NO: 173 - DNA, 44 bp, Artificial Sequence
  Description of Artificial Sequence Synthetic oligonucleotide
gcctccctcgcgccatcagacgctccagggaagctacctggaat

SEQ ID NO: 174 - DNA, 48 bp, Artificial Sequence
  Description of Artificial Sequence Synthetic oligonucleotide
gccttgccagcccgctcagacgctttttaagccaacaaatttgacaac

SEQ ID NO: 175 - DNA, 44 bp, Artificial Sequence
  Description of Artificial Sequence Synthetic oligonucleotide
gcctccctcgcgccatcagacgctagcagccattttgctgattt

SEQ ID NO: 176 - DNA, 47 bp, Artificial Sequence
  Description of Artificial Sequence Synthetic oligonucleotide
gccttgccagcccgctcagacgctaagagataatccaggctttaccc

SEQ ID NO: 177 - DNA, 46 bp, Artificial Sequence
  Description of Artificial Sequence Synthetic oligonucleotide
gcctccctcgcgccatcagacgctaacgtcgtttctatgtgtttcc

SEQ ID NO: 178 - DNA, 44 bp, Artificial Sequence
  Description of Artificial Sequence Synthetic oligonucleotide
gccttgccagcccgctcagacgctgggctgtttgcttcaacaat

SEQ ID NO: 179 - DNA, 47 bp, Artificial Sequence
  Description of Artificial Sequence Synthetic oligonucleotide
gcctccctcgcgccatcagacgcttcatgggaagtacctgttgttct

SEQ ID NO: 180 - DNA, 44 bp, Artificial Sequence
  Description of Artificial Sequence Synthetic oligonucleotide
gccttgccagcccgctcagacgctcgcactgcatgcataagaga

SEQ ID NO: 181 - DNA, 44 bp, Artificial Sequence
  Description of Artificial Sequence Synthetic oligonucleotide
gcctccctcgcgccatcagacgctcagcaagaccctcctttcag

SEQ ID NO: 182 - DNA, 44 bp, Artificial Sequence
  Description of Artificial Sequence Synthetic oligonucleotide
gccttgccagcccgctcagacgctctctgccctctcttggaatg

SEQ ID NO: 183 - DNA, 45 bp, Artificial Sequence
  Description of Artificial Sequence Synthetic oligonucleotide
gcctccctcgcgccatcagacgctaagggttcatttgtttttcca

SEQ ID NO: 184 - DNA, 44 bp, Artificial Sequence
  Description of Artificial Sequence Synthetic oligonucleotide
gccttgccagcccgctcagacgctggagtccaggtcaaggcata

SEQ ID NO: 185 - DNA, 48 bp, Artificial Sequence
  Description of Artificial Sequence Synthetic oligonucleotide
gcctccctcgcgccatcagacgctttggatatgtgcataactgtttga

SEQ ID NO: 186 - DNA, 44 bp, Artificial Sequence
  Description of Artificial Sequence Synthetic oligonucleotide
gccttgccagcccgctcagacgctgtgttgactgtgtgggcgta

SEQ ID NO: 187 - DNA, 48 bp, Artificial Sequence
  Description of Artificial Sequence Synthetic oligonucleotide
gcctccctcgcgccatcagacgcttgaatgctttataagcttgttctg

SEQ ID NO: 188 - DNA, 44 bp, Artificial Sequence
  Description of Artificial Sequence Synthetic oligonucleotide
gccttgccagcccgctcagacgctggtttgtgtgtgtgcctgaa

SEQ ID NO: 189 - DNA, 44 bp, Artificial Sequence
  Description of Artificial Sequence Synthetic oligonucleotide
gcctccctcgcgccatcagacgcttggcttttatccgggtattg

SEQ ID NO: 190 - DNA, 45 bp, Artificial Sequence
  Description of Artificial Sequence Synthetic oligonucleotide
gccttgccagcccgctcagacgctttgggtccaaatgaagactaa

SEQ ID NO: 191 - DNA, 45 bp, Artificial Sequence
  Description of Artificial Sequence Synthetic oligonucleotide
gcctccctcgcgccatcagacgcttctgcatgtcttttctgatgg

SEQ ID NO: 192 - DNA, 43 bp, Artificial Sequence
  Description of Artificial Sequence Synthetic oligonucleotide
gccttgccagcccgctcagacgctctgtcaagggaaggtgcaa

SEQ ID NO: 193 - DNA, 44 bp, Artificial Sequence
  Description of Artificial Sequence Synthetic oligonucleotide
gcctccctcgcgccatcagacgctctgcattcgttctgtgcatc

SEQ ID NO: 194 - DNA, 44 bp, Artificial Sequence
  Description of Artificial Sequence Synthetic oligonucleotide
gccttgccagcccgctcagacgctttgcctcactgagctgtacc

SEQ ID NO: 195 - DNA, 45 bp, Artificial Sequence
  Description of Artificial Sequence Synthetic oligonucleotide
gcctccctcgcgccatcagacgcttgttctggcaatcattgtgaa

SEQ ID NO: 196 - DNA, 45 bp, Artificial Sequence
  Description of Artificial Sequence Synthetic oligonucleotide
gccttgccagcccgctcagacgcttgttttgttcccgtttttctg

SEQ ID NO: 197 - DNA, 44 bp, Artificial Sequence
  Description of Artificial Sequence Synthetic oligonucleotide
gcctccctcgcgccatcagacgctccccgtgtgtctctctctgt

SEQ ID NO: 198 - DNA, 46 bp, Artificial Sequence
  Description of Artificial Sequence Synthetic oligonucleotide
gccttgccagcccgctcagacgctgagaaacagcaaacaagaccaa

SEQ ID NO: 199 - DNA, 44 bp, Artificial Sequence
  Description of Artificial Sequence Synthetic oligonucleotide
gcctccctcgcgccatcagacgctcccaaatcctcatgcattta

SEQ ID NO: 200 - DNA, 44 bp, Artificial Sequence
  Description of Artificial Sequence Synthetic oligonucleotide
gccttgccagcccgctcagacgctaaagcgtgtgaccacctacc

SEQ ID NO: 201 - DNA, 47 bp, Artificial Sequence
  Description of Artificial Sequence Synthetic oligonucleotide
gcctccctcgcgccatcagacgcttgaatcctctttctctcatgtcc

SEQ ID NO: 202 - DNA, 45 bp, Artificial Sequence
  Description of Artificial Sequence Synthetic oligonucleotide
gccttgccagcccgctcagacgcttggagtcatcatgttggagaa

SEQ ID NO: 203 - DNA, 44 bp, Artificial Sequence
  Description of Artificial Sequence Synthetic oligonucleotide
gcctccctcgcgccatcagacgctgcatagagggactcatgctg

SEQ ID NO: 204 - DNA, 49 bp, Artificial Sequence
  Description of Artificial Sequence Synthetic oligonucleotide
gccttgccagcccgctcagacgcttcagatatgtcctgacaaaatgaaa

SEQ ID NO: 205 - DNA, 44 bp, Artificial Sequence
  Description of Artificial Sequence Synthetic oligonucleotide
gcctccctcgcgccatcagacgcttccttgcctttctctcttgc

SEQ ID NO: 206 - DNA, 45 bp, Artificial Sequence
  Description of Artificial Sequence Synthetic oligonucleotide
gccttgccagcccgctcagacgcttttgcacaatggtatgactgc

SEQ ID NO: 207 - DNA, 44 bp, Artificial Sequence
  Description of Artificial Sequence Synthetic oligonucleotide
gcctccctcgcgccatcagacgcttgtgatgcctgaagctgaac

SEQ ID NO: 208 - DNA, 48 bp, Artificial Sequence
  Description of Artificial Sequence Synthetic oligonucleotide
gccttgccagcccgctcagacgctttttctcaaatcactgaaaccaaa

SEQ ID NO: 209 - DNA, 46 bp, Artificial Sequence
  Description of Artificial Sequence Synthetic oligonucleotide
gcctccctcgcgccatcagacgctagcgctactgtgttctctcttg

SEQ ID NO: 210 - DNA, 43 bp, Artificial Sequence
  Description of Artificial Sequence Synthetic oligonucleotide
gccttgccagcccgctcagacgctgcacgctgtgccacctact

SEQ ID NO: 211 - DNA, 44 bp, Artificial Sequence
  Description of Artificial Sequence Synthetic oligonucleotide
gcctccctcgcgccatcagacgctatgttttccagcctgtgacc

SEQ ID NO: 212 - DNA, 44 bp, Artificial Sequence
  Description of Artificial Sequence Synthetic oligonucleotide
gccttgccagcccgctcagacgctcaacatgcatccattcctga

SEQ ID NO: 213 - DNA, 44 bp, Artificial Sequence
  Description of Artificial Sequence Synthetic oligonucleotide
gcctccctcgcgccatcagacgctgcgttcttcgctgtccttac

SEQ ID NO: 214 - DNA, 44 bp, Artificial Sequence
  Description of Artificial Sequence Synthetic oligonucleotide
gccttgccagcccgctcagacgctatgacaaagtccgggttcac

SEQ ID NO: 215 - DNA, 51 bp, Artificial Sequence; identical to SEQ ID NO: 7
  Description of Artificial Sequence Synthetic oligonucleotide

SEQ ID NO: 216 - DNA, 44 bp, Artificial Sequence; identical to SEQ ID NO: 8
  Description of Artificial Sequence Synthetic oligonucleotide

SEQ ID NO: 217 - DNA, 44 bp, Artificial Sequence
  Description of Artificial Sequence Synthetic oligonucleotide
gcctccctcgcgccatcagacgctatccctgctctcatgcagtt

SEQ ID NO: 218 - DNA, 46 bp, Artificial Sequence
  Description of Artificial Sequence Synthetic oligonucleotide
gccttgccagcccgctcagacgcttgagatgctgcattttaaagga

SEQ ID NO: 219 - DNA, 44 bp, Artificial Sequence
  Description of Artificial Sequence Synthetic oligonucleotide
gcctccctcgcgccatcagacgctgctttggctcacctatcacc

SEQ ID NO: 220 - DNA, 49 bp, Artificial Sequence
  Description of Artificial Sequence Synthetic oligonucleotide
gccttgccagcccgctcagacgctcactgaaaggaaatatgacaatgaa

SEQ ID NO: 221 - DNA, 44 bp, Artificial Sequence
  Description of Artificial Sequence Synthetic oligonucleotide
gcctccctcgcgccatcagacgctgccaacatcccacctcttat

SEQ ID NO: 222 - DNA, 44 bp, Artificial Sequence
  Description of Artificial Sequence Synthetic oligonucleotide
gccttgccagcccgctcagacgcttccccctagctgtctgaact

SEQ ID NO: 223 - DNA, 44 bp, Artificial Sequence
  Description of Artificial Sequence Synthetic oligonucleotide
gcctccctcgcgccatcagacgctacggtgctgtgtgttttctg

SEQ ID NO: 224 - DNA, 44 bp, Artificial Sequence
  Description of Artificial Sequence Synthetic oligonucleotide
gccttgccagcccgctcagacgcttggagacatgtggagtgacg

SEQ ID NO: 225 - DNA, 45 bp, Artificial Sequence
  Description of Artificial Sequence Synthetic oligonucleotide
gcctccctcgcgccatcagacgctcggctgtgttctgtactttgc

SEQ ID NO: 226 - DNA, 43 bp, Artificial Sequence
  Description of Artificial Sequence Synthetic oligonucleotide
gccttgccagcccgctcagacgctactctttggcaccaccaca

SEQ ID NO: 227 - DNA, 44 bp, Artificial Sequence
  Description of Artificial Sequence Synthetic oligonucleotide
gcctccctcgcgccatcagacgctatcaacactgtcccacagca

SEQ ID NO: 228 - DNA, 44 bp, Artificial Sequence
  Description of Artificial Sequence Synthetic oligonucleotide
gccttgccagcccgctcagacgctcagctgagccctgttgtaga

SEQ ID NO: 229 - DNA, 44 bp, Artificial Sequence
  Description of Artificial Sequence Synthetic oligonucleotide
gcctccctcgcgccatcagacgcttcatgaaaaaccccaaaacc

SEQ ID NO: 230 - DNA, 45 bp, Artificial Sequence
  Description of Artificial Sequence Synthetic oligonucleotide
gccttgccagcccgctcagacgcttgagtcacacaggtgaggaaa

SEQ ID NO: 231 - DNA, 51 bp, Artificial Sequence
  Description of Artificial Sequence Synthetic oligonucleotide
gcctccctcgcgccatcagacgcttgttacaattctatctgcattgtttct

SEQ ID NO: 232 - DNA, 49 bp, Artificial Sequence
  Description of Artificial Sequence Synthetic oligonucleotide
gccttgccagcccgctcagacgcttgactcagtataacagtggaagctg

SEQ ID NO: 233 - DNA, 48 bp, Artificial Sequence
  Description of Artificial Sequence Synthetic oligonucleotide
gcctccctcgcgccatcagacgctttctagcatttgtctttgcatttc

SEQ ID NO: 234 - DNA, 44 bp, Artificial Sequence
  Description of Artificial Sequence Synthetic oligonucleotide
gccttgccagcccgctcagacgctgagctgcacaaagatgacca

SEQ ID NO: 235 - DNA, 50 bp, Artificial Sequence
  Description of Artificial Sequence Synthetic oligonucleotide
gcctccctcgcgccatcagacgcttgcagatattctcaaacatttaatcc

SEQ ID NO: 236 - DNA, 44 bp, Artificial Sequence
  Description of Artificial Sequence Synthetic oligonucleotide
gccttgccagcccgctcagacgctgcattctttgaccccaagag

SEQ ID NO: 237 - DNA, 44 bp, Artificial Sequence
  Description of Artificial Sequence Synthetic oligonucleotide
gcctccctcgcgccatcagacgctaggacctgcaggactgacat

SEQ ID NO: 238 - DNA, 45 bp, Artificial Sequence
  Description of Artificial Sequence Synthetic oligonucleotide
gccttgccagcccgctcagacgcttgccaacagaataaggcattt

SEQ ID NO: 239 - DNA, 47 bp, Artificial Sequence
  Description of Artificial Sequence Synthetic oligonucleotide
gcctccctcgcgccatcagacgctcattaagtttgcatgtttttcca

SEQ ID NO: 240 - DNA, 44 bp, Artificial Sequence
  Description of Artificial Sequence Synthetic oligonucleotide
gccttgccagcccgctcagacgctggtttcggttctcgttgttg

SEQ ID NO: 241 - DNA, 49 bp, Artificial Sequence
  Description of Artificial Sequence Synthetic oligonucleotide
gcctccctcgcgccatcagacgctgctttattttgtctgtttgtttctt

SEQ ID NO: 242 - DNA, 44 bp, Artificial Sequence
  Description of Artificial Sequence Synthetic oligonucleotide
gccttgccagcccgctcagacgctccccatgcaaagtgatgaac

SEQ ID NO: 243 - DNA, 44 bp, Artificial Sequence
  Description of Artificial Sequence Synthetic oligonucleotide
gcctccctcgcgccatcagacgctagctgtatggcgcttgtttt

SEQ ID NO: 244 - DNA, 49 bp, Artificial Sequence
  Description of Artificial Sequence Synthetic oligonucleotide
gccttgccagcccgctcagacgctcagatattttgacttgagaggttct

SEQ ID NO: 245 - DNA, 44 bp, Artificial Sequence
  Description of Artificial Sequence Synthetic oligonucleotide
gcctccctcgcgccatcagacgctatccttagccagccacctct

SEQ ID NO: 246 - DNA, 44 bp, Artificial Sequence
  Description of Artificial Sequence Synthetic oligonucleotide
gccttgccagcccgctcagacgctacactgcaggaaacaccaca

SEQ ID NO: 247 - DNA, 46 bp, Artificial Sequence
  Description of Artificial Sequence Synthetic oligonucleotide
gcctccctcgcgccatcagacgctcaactgcaagatgaatgttttg

SEQ ID NO: 248 - DNA, 44 bp, Artificial Sequence
  Description of Artificial Sequence Synthetic oligonucleotide
gccttgccagcccgctcagacgcttgtcgtgctgaattcatggt

SEQ ID NO: 249 - DNA, 44 bp, Artificial Sequence
  Description of Artificial Sequence Synthetic oligonucleotide
gcctccctcgcgccatcagacgcttcctggtttgttgcattgac

SEQ ID NO: 250 - DNA, 48 bp, Artificial Sequence
  Description of Artificial Sequence Synthetic oligonucleotide
gccttgccagcccgctcagacgctcaaagaaatgcagaccaatacaaa

SEQ ID NO: 251 - DNA, 47 bp, Artificial Sequence
  Description of Artificial Sequence Synthetic oligonucleotide
gcctccctcgcgccatcagacgctgcaaacttttgctggtacttttc

SEQ ID NO: 252 - DNA, 51 bp, Artificial Sequence
  Description of Artificial Sequence Synthetic oligonucleotide
gccttgccagcccgctcagacgcttcactgctctacaaagattaaagaaac

SEQ ID NO: 253 - DNA, 44 bp, Artificial Sequence
  Description of Artificial Sequence Synthetic oligonucleotide
gcctccctcgcgccatcagacgctatgcatttggtaggtgctca

SEQ ID NO: 254 - DNA, 51 bp, Artificial Sequence
  Description of Artificial Sequence Synthetic oligonucleotide
gccttgccagcccgctcagacgctattaagcaacaaatttcctaatgtagt

SEQ ID NO: 255 - DNA, 45 bp, Artificial Sequence
  Description of Artificial Sequence Synthetic oligonucleotide
gcctccctcgcgccatcagacgcttttctgtgtgtgtctgtctgc

SEQ ID NO: 256 - DNA, 44 bp, Artificial Sequence
  Description of Artificial Sequence Synthetic oligonucleotide
gccttgccagcccgctcagacgctacaaggctggggatgaaaac

SEQ ID NO: 257 - DNA, 49 bp, Artificial Sequence
  Description of Artificial Sequence Synthetic oligonucleotide
gcctccctcgcgccatcagacgctgcctacatttgtttcatttcttttc

SEQ ID NO: 258 - DNA, 43 bp, Artificial Sequence
  Description of Artificial Sequence Synthetic oligonucleotide
gccttgccagcccgctcagacgctccacaaagatgggcagatg

SEQ ID NO: 259 - DNA, 44 bp, Artificial Sequence
  Description of Artificial Sequence Synthetic oligonucleotide
gcctccctcgcgccatcagacgctagcacggtctgatgttttcc

SEQ ID NO: 260 - DNA, 45 bp, Artificial Sequence
  Description of Artificial Sequence Synthetic oligonucleotide
gccttgccagcccgctcagacgctctaggatgccgtgactttcag

SEQ ID NO: 261 - DNA, 49 bp, Artificial Sequence
  Description of Artificial Sequence Synthetic oligonucleotide
gcctccctcgcgccatcagacgctttgatgttcattttattgtttttcc

SEQ ID NO: 262 - DNA, 45 bp, Artificial Sequence
  Description of Artificial Sequence Synthetic oligonucleotide
gccttgccagcccgctcagacgctaagtgccagaacacagatgaa

SEQ ID NO: 263 - DNA, 51 bp, Artificial Sequence
  Description of Artificial Sequence Synthetic oligonucleotide
gcctccctcgcgccatcagacgctttgacttgataataaaagttgtgaatg

SEQ ID NO: 264 - DNA, 44 bp, Artificial Sequence
  Description of Artificial Sequence Synthetic oligonucleotide
gccttgccagcccgctcagacgctcgatgaattcgtccacaaag

SEQ ID NO: 265 - DNA, 46 bp, Artificial Sequence
  Description of Artificial Sequence Synthetic oligonucleotide
gcctccctcgcgccatcagacgctcaatgacgttgacctgtctctc

SEQ ID NO: 266 - DNA, 44 bp, Artificial Sequence
  Description of Artificial Sequence Synthetic oligonucleotide
gccttgccagcccgctcagacgcttctcagagcaaagggctcat

SEQ ID NO: 267 - DNA, 48 bp, Artificial Sequence
  Description of Artificial Sequence Synthetic oligonucleotide
gcctccctcgcgccatcagacgcttggtgtgtgaaatgttttattttc

SEQ ID NO: 268 - DNA, 45 bp, Artificial Sequence
  Description of Artificial Sequence Synthetic oligonucleotide
gccttgccagcccgctcagacgctctctgcacagaaattgggaat

SEQ ID NO: 269 - DNA, 48 bp, Artificial Sequence
  Description of Artificial Sequence Synthetic oligonucleotide
gcctccctcgcgccatcagacgcttggaaggatgtactcaattgtttt

SEQ ID NO: 270 - DNA, 48 bp, Artificial Sequence
  Description of Artificial Sequence Synthetic oligonucleotide
gccttgccagcccgctcagacgcttctgtaattctgtcagttcactcg

SEQ ID NO: 271 - DNA, 44 bp, Artificial Sequence
  Description of Artificial Sequence Synthetic oligonucleotide
gcctccctcgcgccatcagacgctttgtggattgccgtatgtgt

SEQ ID NO: 272 - DNA, 45 bp, Artificial Sequence
  Description of Artificial Sequence Synthetic oligonucleotide
gccttgccagcccgctcagacgctggaccttaaggcttctggcta

SEQ ID NO: 273 - DNA, 44 bp, Artificial Sequence
  Description of Artificial Sequence Synthetic oligonucleotide
gcctccctcgcgccatcagacgctgtggtggtgttcttgtttgc

SEQ ID NO: 274 - DNA, 47 bp, Artificial Sequence
  Description of Artificial Sequence Synthetic oligonucleotide
gccttgccagcccgctcagacgcttctgagtctaccatgatgtctgc

SEQ ID NO: 275 - DNA, 46 bp, Artificial Sequence
  Description of Artificial Sequence Synthetic oligonucleotide
gcctccctcgcgccatcagacgcttgattctttcctgttccctgtt

SEQ ID NO: 276 - DNA, 48 bp, Artificial Sequence
  Description of Artificial Sequence Synthetic oligonucleotide
gccttgccagcccgctcagacgctcacatctgtaaaatccaaagagtc

SEQ ID NO: 277 - DNA, 44 bp, Artificial Sequence
  Description of Artificial Sequence Synthetic oligonucleotide
gcctccctcgcgccatcagacgctgctccggaatgttgatcttg

SEQ ID NO: 278 - DNA, 44 bp, Artificial Sequence
  Description of Artificial Sequence Synthetic oligonucleotide
gccttgccagcccgctcagacgctgcggaacaaattctgctgta

SEQ ID NO: 279 - DNA, 45 bp, Artificial Sequence
  Description of Artificial Sequence Synthetic oligonucleotide
gcctccctcgcgccatcagacgctgaaggctgacttcttcctggt

SEQ ID NO: 280 - DNA, 44 bp, Artificial Sequence
  Description of Artificial Sequence Synthetic oligonucleotide
gccttgccagcccgctcagacgctactcccaggtatccccagag

SEQ ID NO: 281 - DNA, 44 bp, Artificial Sequence
  Description of Artificial Sequence Synthetic oligonucleotide
gcctccctcgcgccatcagacgcttggtgccctcttttcttttt

SEQ ID NO: 282 - DNA, 44 bp, Artificial Sequence
  Description of Artificial Sequence Synthetic oligonucleotide
gccttgccagcccgctcagacgctgaaatcccctttgaacgaat

SEQ ID NO: 283 - DNA, 44 bp, Artificial Sequence
  Description of Artificial Sequence Synthetic oligonucleotide
gcctccctcgcgccatcagacgctactcgggcgtgtactttcag

SEQ ID NO: 284 - DNA, 44 bp, Artificial Sequence
  Description of Artificial Sequence Synthetic oligonucleotide
gccttgccagcccgctcagacgctatgggggtggaagaaatcac

SEQ ID NO: 285 - DNA, 50 bp, Artificial Sequence
  Description of Artificial Sequence Synthetic oligonucleotide
gcctccctcgcgccatcagacgctcaaagtctcaatgtttcctaatgttg

SEQ ID NO: 286 - DNA, 46 bp, Artificial Sequence
  Description of Artificial Sequence Synthetic oligonucleotide
gccttgccagcccgctcagacgctctttggaaaagtatgccactcc

SEQ ID NO: 287 - DNA, 46 bp, Artificial Sequence
  Description of Artificial Sequence Synthetic oligonucleotide
gcctccctcgcgccatcagacgctggagaaaggaaaattgcttcaa

SEQ ID NO: 288 - DNA, 44 bp, Artificial Sequence
  Description of Artificial Sequence Synthetic oligonucleotide
gccttgccagcccgctcagacgctcgtggactaacggattcaga

SEQ ID NO: 289 - DNA, 44 bp, Artificial Sequence
  Description of Artificial Sequence Synthetic oligonucleotide
gcctccctcgcgccatcagacgcttgtgtgtgtgctgacattgg

SEQ ID NO: 290 - DNA, 44 bp, Artificial Sequence
  Description of Artificial Sequence Synthetic oligonucleotide
gccttgccagcccgctcagacgctctgctcacaccatgcgttta

SEQ ID NO: 291 - DNA, 44 bp, Artificial Sequence
  Description of Artificial Sequence Synthetic oligonucleotide
gcctccctcgcgccatcagacgcttctccgtaccacattccaca

SEQ ID NO: 292 - DNA, 45 bp, Artificial Sequence
  Description of Artificial Sequence Synthetic oligonucleotide
gccttgccagcccgctcagacgctcctgtgctttgtcacagacct

SEQ ID NO: 293 - DNA, 47 bp, Artificial Sequence
  Description of Artificial Sequence Synthetic oligonucleotide
gcctccctcgcgccatcagacgctttcatcccttcgtataccttttt

SEQ ID NO: 294 - DNA, 44 bp, Artificial Sequence
  Description of Artificial Sequence Synthetic oligonucleotide
gccttgccagcccgctcagacgcttcattggaaagccaaactga

SEQ ID NO: 295 - DNA, 51 bp, Artificial Sequence
  Description of Artificial Sequence Synthetic oligonucleotide
gcctccctcgcgccatcagacgctttcgtatccatttacaaaatttatcat

SEQ ID NO: 296 - DNA, 46 bp, Artificial Sequence
  Description of Artificial Sequence Synthetic oligonucleotide
gccttgccagcccgctcagacgctctcaaacagatggtgtttctgc

SEQ ID NO: 297 - DNA, 44 bp, Artificial Sequence
  Description of Artificial Sequence Synthetic oligonucleotide
gcctccctcgcgccatcagacgctgtcccagctgctaaatctgc

SEQ ID NO: 298 - DNA, 44 bp, Artificial Sequence
  Description of Artificial Sequence Synthetic oligonucleotide
gccttgccagcccgctcagacgctacatatccacgagcccagac

SEQ ID NO: 299 - DNA, 45 bp, Artificial Sequence
  Description of Artificial Sequence Synthetic oligonucleotide
gcctccctcgcgccatcagacgctcacgtcatactcaatgcctca

SEQ ID NO: 300 - DNA, 44 bp, Artificial Sequence
  Description of Artificial Sequence Synthetic oligonucleotide
gccttgccagcccgctcagacgcttgggggaatgtttagcttga

SEQ ID NO: 301 - DNA, 44 bp, Artificial Sequence
  Description of Artificial Sequence Synthetic oligonucleotide
gcctccctcgcgccatcagcacgagctctccgccttttctctct

SEQ ID NO: 302 - DNA, 44 bp, Artificial Sequence
  Description of Artificial Sequence Synthetic oligonucleotide
gccttgccagcccgctcagcacgagagcggagccaaataatcac

SEQ ID NO: 303 - DNA, 44 bp, Artificial Sequence
  Description of Artificial Sequence Synthetic oligonucleotide
gcctccctcgcgccatcagcacgacgatgctatgagagggaagc

SEQ ID NO: 304 - DNA, 44 bp, Artificial Sequence
  Description of Artificial Sequence Synthetic oligonucleotide
gccttgccagcccgctcagcacgaccaggagcaagtccgtctta

SEQ ID NO: 305 - DNA, 47 bp, Artificial Sequence
  Description of Artificial Sequence Synthetic oligonucleotide
gcctccctcgcgccatcagcacgattttgtgtgtgttttctcttcca

SEQ ID NO: 306 - DNA, 50 bp, Artificial Sequence
  Description of Artificial Sequence Synthetic oligonucleotide
gccttgccagcccgctcagcacgatcataatctgtgtattcaaacagtgc

SEQ ID NO: 307 - DNA, 48 bp, Artificial Sequence
  Description of Artificial Sequence Synthetic oligonucleotide
gcctccctcgcgccatcagcacgaaacaaccaaagctatgtgtatgga

SEQ ID NO: 308 - DNA, 44 bp, Artificial Sequence
  Description of Artificial Sequence Synthetic oligonucleotide
gccttgccagcccgctcagcacgacatgtgcaaaacacaaccaa

SEQ ID NO: 309 - DNA, 48 bp, Artificial Sequence
  Description of Artificial Sequence Synthetic oligonucleotide
gcctccctcgcgccatcagcacgatttctcctttcttttctttttcca

SEQ ID NO: 310 - DNA, 44 bp, Artificial Sequence
  Description of Artificial Sequence Synthetic oligonucleotide
gccttgccagcccgctcagcacgaagcatctccaaaaccattgc

SEQ ID NO: 311 - DNA, 45 bp, Artificial Sequence
  Description of Artificial Sequence Synthetic oligonucleotide
gcctccctcgcgccatcagcacgaccatcctgaaatgtgatgctt

SEQ ID NO: 312 - DNA, 44 bp, Artificial Sequence
  Description of Artificial Sequence Synthetic oligonucleotide
gccttgccagcccgctcagcacgatcctttgtggcatctcttcc

SEQ ID NO: 313 - DNA, 45 bp, Artificial Sequence
  Description of Artificial Sequence Synthetic oligonucleotide
gcctccctcgcgccatcagcacgaggacatttgctctgttggttt

SEQ ID NO: 314 - DNA, 47 bp, Artificial Sequence
  Description of Artificial Sequence Synthetic oligonucleotide
gccttgccagcccgctcagcacgattcatggctagaaaggatcaaaa

SEQ ID NO: 315 - DNA, 44 bp, Artificial Sequence
  Description of Artificial Sequence Synthetic oligonucleotide
gcctccctcgcgccatcagcacgagactggaagtgctgtgtgga

SEQ ID NO: 316 - DNA, 44 bp, Artificial Sequence
  Description of Artificial Sequence Synthetic oligonucleotide
gccttgccagcccgctcagcacgatgggatcgcttcttaactgc

SEQ ID NO: 317 - DNA, 46 bp, Artificial Sequence
  Description of Artificial Sequence Synthetic oligonucleotide
gcctccctcgcgccatcagcacgacagcattttgacaatgtttctg

SEQ ID NO: 318 - DNA, 49 bp, Artificial Sequence
  Description of Artificial Sequence Synthetic oligonucleotide
gccttgccagcccgctcagcacgatctttacctcaaaagaacaaaagag

SEQ ID NO: 319 - DNA, 44 bp, Artificial Sequence
  Description of Artificial Sequence Synthetic oligonucleotide
gcctccctcgcgccatcagcacgaccagggaagctacctggaat

SEQ ID NO: 320 - DNA, 48 bp, Artificial Sequence
  Description of Artificial Sequence Synthetic oligonucleotide
gccttgccagcccgctcagcacgattttaagccaacaaatttgacaac

SEQ ID NO: 321 - DNA, 44 bp, Artificial Sequence
  Description of Artificial Sequence Synthetic oligonucleotide
gcctccctcgcgccatcagcacgaagcagccattttgctgattt

SEQ ID NO: 322 - DNA, 47 bp, Artificial Sequence
  Description of Artificial Sequence Synthetic oligonucleotide
gccttgccagcccgctcagcacgaaagagataatccaggctttaccc

SEQ ID NO: 323 - DNA, 46 bp, Artificial Sequence
  Description of Artificial Sequence Synthetic oligonucleotide
gcctccctcgcgccatcagcacgaaacgtcgtttctatgtgtttcc

SEQ ID NO: 324 - DNA, 44 bp, Artificial Sequence
  Description of Artificial Sequence Synthetic oligonucleotide
gccttgccagcccgctcagcacgagggctgtttgcttcaacaat

SEQ ID NO: 325 - DNA, 47 bp, Artificial Sequence
  Description of Artificial Sequence Synthetic oligonucleotide
gcctccctcgcgccatcagcacgatcatgggaagtacctgttgttct

SEQ ID NO: 326 - DNA, 44 bp, Artificial Sequence
  Description of Artificial Sequence Synthetic oligonucleotide
gccttgccagcccgctcagcacgacgcactgcatgcataagaga

SEQ ID NO: 327 - DNA, 44 bp, Artificial Sequence
  Description of Artificial Sequence Synthetic oligonucleotide
gcctccctcgcgccatcagcacgacagcaagaccctcctttcag

SEQ ID NO: 328 - DNA, 44 bp, Artificial Sequence
  Description of Artificial Sequence Synthetic oligonucleotide
gccttgccagcccgctcagcacgactctgccctctcttggaatg

SEQ ID NO: 329 - DNA, 45 bp, Artificial Sequence
  Description of Artificial Sequence Synthetic oligonucleotide
gcctccctcgcgccatcagcacgaaagggttcatttgtttttcca

SEQ ID NO: 330 - DNA, 44 bp, Artificial Sequence
  Description of Artificial Sequence Synthetic oligonucleotide
gccttgccagcccgctcagcacgaggagtccaggtcaaggcata

SEQ ID NO: 331 - DNA, 48 bp, Artificial Sequence
  Description of Artificial Sequence Synthetic oligonucleotide
gcctccctcgcgccatcagcacgattggatatgtgcataactgtttga

SEQ ID NO: 332 - DNA, 44 bp, Artificial Sequence
  Description of Artificial Sequence Synthetic oligonucleotide
gccttgccagcccgctcagcacgagtgttgactgtgtgggcgta

SEQ ID NO: 333 - DNA, 48 bp, Artificial Sequence
  Description of Artificial Sequence Synthetic oligonucleotide
gcctccctcgcgccatcagcacgatgaatgctttataagcttgttctg

SEQ ID NO: 334 - DNA, 44 bp, Artificial Sequence
  Description of Artificial Sequence Synthetic oligonucleotide
gccttgccagcccgctcagcacgaggtttgtgtgtgtgcctgaa

SEQ ID NO: 335 - DNA, 44 bp, Artificial Sequence
  Description of Artificial Sequence Synthetic oligonucleotide
gcctccctcgcgccatcagcacgatggcttttatccgggtattg

SEQ ID NO: 336 - DNA, 45 bp, Artificial Sequence
  Description of Artificial Sequence Synthetic oligonucleotide
gccttgccagcccgctcagcacgattgggtccaaatgaagactaa

SEQ ID NO: 337 - DNA, 45 bp, Artificial Sequence
  Description of Artificial Sequence Synthetic oligonucleotide
gcctccctcgcgccatcagcacgatctgcatgtcttttctgatgg

SEQ ID NO: 338 - DNA, 43 bp, Artificial Sequence
  Description of Artificial Sequence Synthetic oligonucleotide
gccttgccagcccgctcagcacgactgtcaagggaaggtgcaa

SEQ ID NO: 339 - DNA, 44 bp, Artificial Sequence
  Description of Artificial Sequence Synthetic oligonucleotide
gcctccctcgcgccatcagcacgactgcattcgttctgtgcatc

SEQ ID NO: 340 - DNA, 44 bp, Artificial Sequence
  Description of Artificial Sequence Synthetic oligonucleotide
gccttgccagcccgctcagcacgattgcctcactgagctgtacc

SEQ ID NO: 341 - DNA, 45 bp, Artificial Sequence
  Description of Artificial Sequence Synthetic oligonucleotide
gcctccctcgcgccatcagcacgatgttctggcaatcattgtgaa

SEQ ID NO: 342 - DNA, 45 bp, Artificial Sequence
  Description of Artificial Sequence Synthetic oligonucleotide
gccttgccagcccgctcagcacgatgttttgttcccgtttttctg

SEQ ID NO: 343 - DNA, 44 bp, Artificial Sequence
  Description of Artificial Sequence Synthetic oligonucleotide
gcctccctcgcgccatcagcacgaccccgtgtgtctctctctgt

SEQ ID NO: 344 - DNA, 46 bp, Artificial Sequence
  Description of Artificial Sequence Synthetic oligonucleotide
gccttgccagcccgctcagcacgagagaaacagcaaacaagaccaa

SEQ ID NO: 345 - DNA, 44 bp, Artificial Sequence
  Description of Artificial Sequence Synthetic oligonucleotide
gcctccctcgcgccatcagcacgacccaaatcctcatgcattta

SEQ ID NO: 346 - DNA, 44 bp, Artificial Sequence
  Description of Artificial Sequence Synthetic oligonucleotide
gccttgccagcccgctcagcacgaaaagcgtgtgaccacctacc

SEQ ID NO: 347 - DNA, 47 bp, Artificial Sequence
  Description of Artificial Sequence Synthetic oligonucleotide
gcctccctcgcgccatcagcacgatgaatcctctttctctcatgtcc

SEQ ID NO: 348 - DNA, 45 bp, Artificial Sequence
  Description of Artificial Sequence Synthetic oligonucleotide
gccttgccagcccgctcagcacgatggagtcatcatgttggagaa

SEQ ID NO: 349 - DNA, 44 bp, Artificial Sequence
  Description of Artificial Sequence Synthetic oligonucleotide
gcctccctcgcgccatcagcacgagcatagagggactcatgctg

SEQ ID NO: 350 - DNA, 49 bp, Artificial Sequence
  Description of Artificial Sequence Synthetic oligonucleotide
gccttgccagcccgctcagcacgatcagatatgtcctgacaaaatgaaa

SEQ ID NO: 351 - DNA, 44 bp, Artificial Sequence
  Description of Artificial Sequence Synthetic oligonucleotide
gcctccctcgcgccatcagcacgatccttgcctttctctcttgc

SEQ ID NO: 352 - DNA, 45 bp, Artificial Sequence
  Description of Artificial Sequence Synthetic oligonucleotide
gccttgccagcccgctcagcacgatttgcacaatggtatgactgc

SEQ ID NO: 353 - DNA, 44 bp, Artificial Sequence
  Description of Artificial Sequence Synthetic oligonucleotide
gcctccctcgcgccatcagcacgatgtgatgcctgaagctgaac

SEQ ID NO: 354 - DNA, 48 bp, Artificial Sequence
  Description of Artificial Sequence Synthetic oligonucleotide
gccttgccagcccgctcagcacgattttctcaaatcactgaaaccaaa

SEQ ID NO: 355 - DNA, 46 bp, Artificial Sequence
  Description of Artificial Sequence Synthetic oligonucleotide
gcctccctcgcgccatcagcacgaagcgctactgtgttctctcttg

SEQ ID NO: 356 - DNA, 43 bp, Artificial Sequence
  Description of Artificial Sequence Synthetic oligonucleotide
gccttgccagcccgctcagcacgagcacgctgtgccacctact

SEQ ID NO: 357 - DNA, 44 bp, Artificial Sequence
  Description of Artificial Sequence Synthetic oligonucleotide
gcctccctcgcgccatcagcacgaatgttttccagcctgtgacc

SEQ ID NO: 358 - DNA, 44 bp, Artificial Sequence
  Description of Artificial Sequence Synthetic oligonucleotide
gccttgccagcccgctcagcacgacaacatgcatccattcctga

SEQ ID NO: 359 - DNA, 44 bp, Artificial Sequence
  Description of Artificial Sequence Synthetic oligonucleotide
gcctccctcgcgccatcagcacgagcgttcttcgctgtccttac

SEQ ID NO: 360 - DNA, 44 bp, Artificial Sequence
  Description of Artificial Sequence Synthetic oligonucleotide
gccttgccagcccgctcagcacgaatgacaaagtccgggttcac

SEQ ID NO: 361 - DNA, 51 bp, Artificial Sequence
  Description of Artificial Sequence Synthetic oligonucleotide
gcctccctcgcgccatcagcacgagccgtatgacataattactattctttt

SEQ ID NO: 362 - DNA, 44 bp, Artificial Sequence
  Description of Artificial Sequence Synthetic oligonucleotide
gccttgccagcccgctcagcacgacccactcaaattcaggcaat

SEQ ID NO: 363 - DNA, 44 bp, Artificial Sequence
  Description of Artificial Sequence Synthetic oligonucleotide
gcctccctcgcgccatcagcacgaatccctgctctcatgcagtt

SEQ ID NO: 364 - DNA, 46 bp, Artificial Sequence
  Description of Artificial Sequence Synthetic oligonucleotide
gccttgccagcccgctcagcacgatgagatgctgcattttaaagga

SEQ ID NO: 365 - DNA, 44 bp, Artificial Sequence
  Description of Artificial Sequence Synthetic oligonucleotide
gcctccctcgcgccatcagcacgagctttggctcacctatcacc

SEQ ID NO: 366 - DNA, 49 bp, Artificial Sequence
  Description of Artificial Sequence Synthetic oligonucleotide
gccttgccagcccgctcagcacgacactgaaaggaaatatgacaatgaa

SEQ ID NO: 367 - DNA, 44 bp, Artificial Sequence
  Description of Artificial Sequence Synthetic oligonucleotide
gcctccctcgcgccatcagcacgagccaacatcccacctcttat

SEQ ID NO: 368 - DNA, 44 bp, Artificial Sequence
  Description of Artificial Sequence Synthetic oligonucleotide
gccttgccagcccgctcagcacgatccccctagctgtctgaact

SEQ ID NO: 369 - DNA, 44 bp, Artificial Sequence
  Description of Artificial Sequence Synthetic oligonucleotide
gcctccctcgcgccatcagcacgaacggtgctgtgtgttttctg

SEQ ID NO: 370 - DNA, 44 bp, Artificial Sequence
  Description of Artificial Sequence Synthetic oligonucleotide
gccttgccagcccgctcagcacgatggagacatgtggagtgacg

SEQ ID NO: 371 - DNA, 45 bp, Artificial Sequence
  Description of Artificial Sequence Synthetic oligonucleotide
gcctccctcgcgccatcagcacgacggctgtgttctgtactttgc

SEQ ID NO: 372 - DNA, 43 bp, Artificial Sequence
  Description of Artificial Sequence Synthetic oligonucleotide
gccttgccagcccgctcagcacgaactctttggcaccaccaca

SEQ ID NO: 373 - DNA, 44 bp, Artificial Sequence
  Description of Artificial Sequence Synthetic oligonucleotide
gcctccctcgcgccatcagcacgaatcaacactgtcccacagca

SEQ ID NO: 374 - DNA, 44 bp, Artificial Sequence
  Description of Artificial Sequence Synthetic oligonucleotide
gccttgccagcccgctcagcacgacagctgagccctgttgtaga

SEQ ID NO: 375 - DNA, 44 bp, Artificial Sequence
  Description of Artificial Sequence Synthetic oligonucleotide
gcctccctcgcgccatcagcacgatcatgaaaaaccccaaaacc

SEQ ID NO: 376 - DNA, 45 bp, Artificial Sequence
  Description of Artificial Sequence Synthetic oligonucleotide
gccttgccagcccgctcagcacgatgagtcacacaggtgaggaaa

SEQ ID NO: 377 - DNA, 51 bp, Artificial Sequence
  Description of Artificial Sequence Synthetic oligonucleotide
gcctccctcgcgccatcagcacgatgttacaattctatctgcattgtttct

SEQ ID NO: 378 - DNA, 49 bp, Artificial Sequence
  Description of Artificial Sequence Synthetic oligonucleotide
gccttgccagcccgctcagcacgatgactcagtataacagtggaagctg

SEQ ID NO: 379 - DNA, 48 bp, Artificial Sequence
  Description of Artificial Sequence Synthetic oligonucleotide
gcctccctcgcgccatcagcacgattctagcatttgtctttgcatttc

SEQ ID NO: 380 - DNA, 44 bp, Artificial Sequence
  Description of Artificial Sequence Synthetic oligonucleotide
gccttgccagcccgctcagcacgagagctgcacaaagatgacca

SEQ ID NO: 381 - DNA, 50 bp, Artificial Sequence
  Description of Artificial Sequence Synthetic oligonucleotide
gcctccctcgcgccatcagcacgatgcagatattctcaaacatttaatcc

SEQ ID NO: 382 - DNA, 44 bp, Artificial Sequence
  Description of Artificial Sequence Synthetic oligonucleotide
gccttgccagcccgctcagcacgagcattctttgaccccaagag

SEQ ID NO: 383 - DNA, 44 bp, Artificial Sequence
  Description of Artificial Sequence Synthetic oligonucleotide
gcctccctcgcgccatcagcacgaaggacctgcaggactgacat

SEQ ID NO: 384 - DNA, 45 bp, Artificial Sequence
  Description of Artificial Sequence Synthetic oligonucleotide
gccttgccagcccgctcagcacgatgccaacagaataaggcattt

SEQ ID NO: 385 - DNA, 47 bp, Artificial Sequence
  Description of Artificial Sequence Synthetic oligonucleotide
gcctccctcgcgccatcagcacgacattaagtttgcatgtttttcca

SEQ ID NO: 386 - DNA, 44 bp, Artificial Sequence
  Description of Artificial Sequence Synthetic oligonucleotide
gccttgccagcccgctcagcacgaggtttcggttctcgttgttg

SEQ ID NO: 387 - DNA, 49 bp, Artificial Sequence
  Description of Artificial Sequence Synthetic oligonucleotide
gcctccctcgcgccatcagcacgagctttattttgtctgtttgtttctt

SEQ ID NO: 388 - DNA, 44 bp, Artificial Sequence
  Description of Artificial Sequence Synthetic oligonucleotide
gccttgccagcccgctcagcacgaccccatgcaaagtgatgaac

SEQ ID NO: 389 - DNA, 44 bp, Artificial Sequence
  Description of Artificial Sequence Synthetic oligonucleotide
gcctccctcgcgccatcagcacgaagctgtatggcgcttgtttt

SEQ ID NO: 390 - DNA, 49 bp, Artificial Sequence
  Description of Artificial Sequence Synthetic oligonucleotide
gccttgccagcccgctcagcacgacagatattttgacttgagaggttct

SEQ ID NO: 391 - DNA, 44 bp, Artificial Sequence
  Description of Artificial Sequence Synthetic oligonucleotide
gcctccctcgcgccatcagcacgaatccttagccagccacctct

SEQ ID NO: 392 - DNA, 44 bp, Artificial Sequence
  Description of Artificial Sequence Synthetic oligonucleotide
gccttgccagcccgctcagcacgaacactgcaggaaacaccaca

SEQ ID NO: 393 - DNA, 46 bp, Artificial Sequence
  Description of Artificial Sequence Synthetic oligonucleotide
gcctccctcgcgccatcagcacgacaactgcaagatgaatgttttg

SEQ ID NO: 394 - DNA, 44 bp, Artificial Sequence
  Description of Artificial Sequence Synthetic oligonucleotide
gccttgccagcccgctcagcacgatgtcgtgctgaattcatggt

SEQ ID NO: 395 - DNA, 44 bp, Artificial Sequence
  Description of Artificial Sequence Synthetic oligonucleotide
gcctccctcgcgccatcagcacgatcctggtttgttgcattgac

SEQ ID NO: 396 - DNA, 48 bp, Artificial Sequence
  Description of Artificial Sequence Synthetic oligonucleotide
gccttgccagcccgctcagcacgacaaagaaatgcagaccaatacaaa

SEQ ID NO: 397 - DNA, 47 bp, Artificial Sequence
  Description of Artificial Sequence Synthetic oligonucleotide
gcctccctcgcgccatcagcacgagcaaacttttgctggtacttttc

SEQ ID NO: 398 - DNA, 51 bp, Artificial Sequence
  Description of Artificial Sequence Synthetic oligonucleotide
gccttgccagcccgctcagcacgatcactgctctacaaagattaaagaaac

SEQ ID NO: 399 - DNA, 44 bp, Artificial Sequence
  Description of Artificial Sequence Synthetic oligonucleotide
gcctccctcgcgccatcagcacgaatgcatttggtaggtgctca

SEQ ID NO: 400 - DNA, 51 bp, Artificial Sequence
  Description of Artificial Sequence Synthetic oligonucleotide
gccttgccagcccgctcagcacgaattaagcaacaaatttcctaatgtagt

SEQ ID NO: 401 - DNA, 45 bp, Artificial Sequence
  Description of Artificial Sequence Synthetic oligonucleotide
gcctccctcgcgccatcagcacgatttctgtgtgtgtctgtctgc

SEQ ID NO: 402 - DNA, 44 bp, Artificial Sequence
  Description of Artificial Sequence Synthetic oligonucleotide
gccttgccagcccgctcagcacgaacaaggctggggatgaaaac

SEQ ID NO: 403 - DNA, 49 bp, Artificial Sequence
  Description of Artificial Sequence Synthetic oligonucleotide
gcctccctcgcgccatcagcacgagcctacatttgtttcatttcttttc

SEQ ID NO: 404 - DNA, 43 bp, Artificial Sequence
  Description of Artificial Sequence Synthetic oligonucleotide
gccttgccagcccgctcagcacgaccacaaagatgggcagatg

SEQ ID NO: 405 - DNA, 44 bp, Artificial Sequence
  Description of Artificial Sequence Synthetic oligonucleotide
gcctccctcgcgccatcagcacgaagcacggtctgatgttttcc

SEQ ID NO: 406 - DNA, 45 bp, Artificial Sequence
  Description of Artificial Sequence Synthetic oligonucleotide
gccttgccagcccgctcagcacgactaggatgccgtgactttcag

SEQ ID NO: 407 - DNA, 49 bp, Artificial Sequence
  Description of Artificial Sequence Synthetic oligonucleotide
gcctccctcgcgccatcagcacgattgatgttcattttattgtttttcc

SEQ ID NO: 408 - DNA, 45 bp, Artificial Sequence
  Description of Artificial Sequence Synthetic oligonucleotide
gccttgccagcccgctcagcacgaaagtgccagaacacagatgaa

SEQ ID NO: 409 - DNA, 51 bp, Artificial Sequence
  Description of Artificial Sequence Synthetic oligonucleotide
gcctccctcgcgccatcagcacgattgacttgataataaaagttgtgaatg

SEQ ID NO: 410 - DNA, 44 bp, Artificial Sequence
  Description of Artificial Sequence Synthetic oligonucleotide
gccttgccagcccgctcagcacgacgatgaattcgtccacaaag

SEQ ID NO: 411 - DNA, 46 bp, Artificial Sequence
  Description of Artificial Sequence Synthetic oligonucleotide
gcctccctcgcgccatcagcacgacaatgacgttgacctgtctctc

SEQ ID NO: 412 - DNA, 44 bp, Artificial Sequence
  Description of Artificial Sequence Synthetic oligonucleotide
gccttgccagcccgctcagcacgatctcagagcaaagggctcat

SEQ ID NO: 413 - DNA, 48 bp, Artificial Sequence
  Description of Artificial Sequence Synthetic oligonucleotide
gcctccctcgcgccatcagcacgatggtgtgtgaaatgttttattttc

SEQ ID NO: 414 - DNA, 45 bp, Artificial Sequence
  Description of Artificial Sequence Synthetic oligonucleotide
gccttgccagcccgctcagcacgactctgcacagaaattgggaat

SEQ ID NO: 415 - DNA, 48 bp, Artificial Sequence
  Description of Artificial Sequence Synthetic oligonucleotide
gcctccctcgcgccatcagcacgatggaaggatgtactcaattgtttt

SEQ ID NO: 416 - DNA, 48 bp, Artificial Sequence
  Description of Artificial Sequence Synthetic oligonucleotide
gccttgccagcccgctcagcacgatctgtaattctgtcagttcactcg

SEQ ID NO: 417 - DNA, 44 bp, Artificial Sequence
  Description of Artificial Sequence Synthetic oligonucleotide
gcctccctcgcgccatcagcacgattgtggattgccgtatgtgt

SEQ ID NO: 418 - DNA, 45 bp, Artificial Sequence
  Description of Artificial Sequence Synthetic oligonucleotide
gccttgccagcccgctcagcacgaggaccttaaggcttctggcta

SEQ ID NO: 419 - DNA, 44 bp, Artificial Sequence
  Description of Artificial Sequence Synthetic oligonucleotide
gcctccctcgcgccatcagcacgagtggtggtgttcttgtttgc

SEQ ID NO: 420 - DNA, 47 bp, Artificial Sequence
  Description of Artificial Sequence Synthetic oligonucleotide
gccttgccagcccgctcagcacgatctgagtctaccatgatgtctgc

SEQ ID NO: 421 - DNA, 46 bp, Artificial Sequence
  Description of Artificial Sequence Synthetic oligonucleotide
gcctccctcgcgccatcagcacgatgattctttcctgttccctgtt

SEQ ID NO: 422 - DNA, 48 bp, Artificial Sequence
  Description of Artificial Sequence Synthetic oligonucleotide
gccttgccagcccgctcagcacgacacatctgtaaaatccaaagagtc

SEQ ID NO: 423 - DNA, 44 bp, Artificial Sequence
  Description of Artificial Sequence Synthetic oligonucleotide
gcctccctcgcgccatcagcacgagctccggaatgttgatcttg

SEQ ID NO: 424 - DNA, 44 bp, Artificial Sequence
  Description of Artificial Sequence Synthetic oligonucleotide
gccttgccagcccgctcagcacgagcggaacaaattctgctgta

SEQ ID NO: 425 - DNA, 45 bp, Artificial Sequence
  Description of Artificial Sequence Synthetic oligonucleotide
gcctccctcgcgccatcagcacgagaaggctgacttcttcctggt

SEQ ID NO: 426 - DNA, 44 bp, Artificial Sequence
  Description of Artificial Sequence Synthetic oligonucleotide
gccttgccagcccgctcagcacgaactcccaggtatccccagag

SEQ ID NO: 427 - DNA, 44 bp, Artificial Sequence
  Description of Artificial Sequence Synthetic oligonucleotide
gcctccctcgcgccatcagcacgatggtgccctcttttcttttt

SEQ ID NO: 428 - DNA, 44 bp, Artificial Sequence
  Description of Artificial Sequence Synthetic oligonucleotide
gccttgccagcccgctcagcacgagaaatcccctttgaacgaat

SEQ ID NO: 429 - DNA, 44 bp, Artificial Sequence
  Description of Artificial Sequence Synthetic oligonucleotide
gcctccctcgcgccatcagcacgaactcgggcgtgtactttcag

SEQ ID NO: 430 - DNA, 44 bp, Artificial Sequence
  Description of Artificial Sequence Synthetic oligonucleotide
gccttgccagcccgctcagcacgaatgggggtggaagaaatcac

SEQ ID NO: 431 - DNA, 50 bp, Artificial Sequence
  Description of Artificial Sequence Synthetic oligonucleotide
gcctccctcgcgccatcagcacgacaaagtctcaatgtttcctaatgttg

SEQ ID NO: 432 - DNA, 46 bp, Artificial Sequence
  Description of Artificial Sequence Synthetic oligonucleotide
gccttgccagcccgctcagcacgactttggaaaagtatgccactcc

SEQ ID NO: 433 - DNA, 46 bp, Artificial Sequence
  Description of Artificial Sequence Synthetic oligonucleotide
gcctccctcgcgccatcagcacgaggagaaaggaaaattgcttcaa

SEQ ID NO: 434 - DNA, 44 bp, Artificial Sequence
  Description of Artificial Sequence Synthetic oligonucleotide
gccttgccagcccgctcagcacgacgtggactaacggattcaga

SEQ ID NO: 435 - DNA, 44 bp, Artificial Sequence
  Description of Artificial Sequence Synthetic oligonucleotide
gcctccctcgcgccatcagcacgatgtgtgtgtgctgacattgg

SEQ ID NO: 436 - DNA, 44 bp, Artificial Sequence
  Description of Artificial Sequence Synthetic oligonucleotide
gccttgccagcccgctcagcacgactgctcacaccatgcgttta

SEQ ID NO: 437 - DNA, 44 bp, Artificial Sequence
  Description of Artificial Sequence Synthetic oligonucleotide
gcctccctcgcgccatcagcacgatctccgtaccacattccaca

SEQ ID NO: 438 - DNA, 45 bp, Artificial Sequence
  Description of Artificial Sequence Synthetic oligonucleotide
gccttgccagcccgctcagcacgacctgtgctttgtcacagacct

SEQ ID NO: 439 - DNA, 47 bp, Artificial Sequence
  Description of Artificial Sequence Synthetic oligonucleotide
gcctccctcgcgccatcagcacgattcatcccttcgtataccttttt

SEQ ID NO: 440 - DNA, 44 bp, Artificial Sequence
  Description of Artificial Sequence Synthetic oligonucleotide
gccttgccagcccgctcagcacgatcattggaaagccaaactga

SEQ ID NO: 441 - DNA, 51 bp, Artificial Sequence
  Description of Artificial Sequence Synthetic oligonucleotide
gcctccctcgcgccatcagcacgattcgtatccatttacaaaatttatcat

SEQ ID NO: 442 - DNA, 46 bp, Artificial Sequence
  Description of Artificial Sequence Synthetic oligonucleotide
gccttgccagcccgctcagcacgactcaaacagatggtgtttctgc

SEQ ID NO: 443 - DNA, 44 bp, Artificial Sequence
  Description of Artificial Sequence Synthetic oligonucleotide
gcctccctcgcgccatcagcacgagtcccagctgctaaatctgc

SEQ ID NO: 444 - DNA, 44 bp, Artificial Sequence
  Description of Artificial Sequence Synthetic oligonucleotide
gccttgccagcccgctcagcacgaacatatccacgagcccagac

SEQ ID NO: 445 - DNA, 45 bp, Artificial Sequence
  Description of Artificial Sequence Synthetic oligonucleotide
gcctccctcgcgccatcagcacgacacgtcatactcaatgcctca

SEQ ID NO: 446 - DNA, 44 bp, Artificial Sequence
  Description of Artificial Sequence Synthetic oligonucleotide
gccttgccagcccgctcagcacgatgggggaatgtttagcttga

SEQ ID NO: 447 - DNA, 24 bp, Artificial Sequence
  Description of Artificial Sequence Synthetic oligonucleotide
gcctccctcgcgccatcagacgct

SEQ ID NO: 448 - DNA, 24 bp, Artificial Sequence
  Description of Artificial Sequence Synthetic oligonucleotide
gccttgccagcccgctcagacgct

SEQ ID NO: 449 - DNA, 24 bp, Artificial Sequence
  Description of Artificial Sequence Synthetic oligonucleotide
gcctccctcgcgccatcagcacga

SEQ ID NO: 450 - DNA, 24 bp, Artificial Sequence
  Description of Artificial Sequence Synthetic oligonucleotide
gccttgccagcccgctcagcacga

SEQ ID NO: 451 - DNA, 21 bp, Artificial Sequence
  Description of Artificial Sequence Synthetic oligonucleotide
tcttaagcgtcgatggaggag

SEQ ID NO: 452 - DNA, 23 bp, Artificial Sequence
  Description of Artificial Sequence Synthetic oligonucleotide
ttgaaacccaaggtacatttcag

SEQ ID NO: 453 - DNA, 21 bp, Artificial Sequence
  Description of Artificial Sequence Synthetic oligonucleotide
cgtcatctttggagcaggaac

SEQ ID NO: 454 - DNA, 21 bp, Artificial Sequence
  Description of Artificial Sequence Synthetic oligonucleotide
atgcatggcattagcaaagac

SEQ ID NO: 455 - DNA, 27 bp, Artificial Sequence
  Description of Artificial Sequence Synthetic oligonucleotide
tggtgtagtggaaactaggaattacat

SEQ ID NO: 456 - DNA, 21 bp, Artificial Sequence
  Description of Artificial Sequence Synthetic oligonucleotide
tggattaagaagcaatgccct

SEQ ID NO: 457 - DNA, 24 bp, Artificial Sequence
  Description of Artificial Sequence Synthetic oligonucleotide
tcagttgcctgaagagaaacataa

SEQ ID NO: 458 - DNA, 21 bp, Artificial Sequence
  Description of Artificial Sequence Synthetic oligonucleotide
taacagtctgcatggagcagg

SEQ ID NO: 459 - DNA, 22 bp, Artificial Sequence
  Description of Artificial Sequence Synthetic oligonucleotide
gaacaaaccaggattctagccc

SEQ ID NO: 460 - DNA, 21 bp, Artificial Sequence
  Description of Artificial Sequence Synthetic oligonucleotide
agtggttgccaccttgttacc

SEQ ID NO: 461 - DNA, 18 bp, Artificial Sequence
  Description of Artificial Sequence Synthetic oligonucleotide
agctctccgcctcccttc

SEQ ID NO: 462 - DNA, 18 bp, Artificial Sequence
  Description of Artificial Sequence Synthetic oligonucleotide
aaggtggctgagggcatc

SEQ ID NO: 463 - DNA, 23 bp, Artificial Sequence
  Description of Artificial Sequence Synthetic oligonucleotide
gaggaacactggcagttactgtg

SEQ ID NO: 464 - DNA, 26 bp, Artificial Sequence
  Description of Artificial Sequence Synthetic oligonucleotide
tctcttcccaaatctattcctaatcc

SEQ ID NO: 465 - DNA, 20 bp, Artificial Sequence
  Description of Artificial Sequence Synthetic oligonucleotide
aggacaaagtccggattgaa

SEQ ID NO: 466 - DNA, 22 bp, Artificial Sequence
  Description of Artificial Sequence Synthetic oligonucleotide
caaagaaacagcaaaatggtga

SEQ ID NO: 467 - DNA, 23 bp, Artificial Sequence
  Description of Artificial Sequence Synthetic oligonucleotide
ttgctccctttacctcttatcaa

SEQ ID NO: 468 - DNA, 21 bp, Artificial Sequence
  Description of Artificial Sequence Synthetic oligonucleotide
tttcacgtcacatacaaacca

SEQ ID NO: 469 - DNA, 23 bp, Artificial Sequence
  Description of Artificial Sequence Synthetic oligonucleotide
ccaagaaaggcttgtgtctacat

SEQ ID NO: 470 - DNA, 24 bp, Artificial Sequence
  Description of Artificial Sequence Synthetic oligonucleotide
tcatccatatttcacattccctaa

SEQ ID NO: 471 - DNA, 24 bp, Artificial Sequence
  Description of Artificial Sequence Synthetic oligonucleotide
tgtgagtttttgaaatctctgtga

SEQ ID NO: 472 - DNA, 27 bp, Artificial Sequence
  Description of Artificial Sequence Synthetic oligonucleotide
tggtaggtagaaaagagatatttttgg

SEQ ID NO: 473 - DNA, 20 bp, Artificial Sequence
  Description of Artificial Sequence Synthetic oligonucleotide
gaagcttctgggttttgcac

SEQ ID NO: 474 - DNA, 20 bp, Artificial Sequence
  Description of Artificial Sequence Synthetic oligonucleotide
ccaaaagaaagcggttcaag

SEQ ID NO: 475 - DNA, 26 bp, Artificial Sequence
  Description of Artificial Sequence Synthetic oligonucleotide
tcttatcttccaatgacttcattttt

SEQ ID NO: 476 - DNA, 27 bp, Artificial Sequence
  Description of Artificial Sequence Synthetic oligonucleotide
gcagaaaaataaagatacatacttggt

SEQ ID NO: 477 - DNA, 24 bp, Artificial Sequence
  Description of Artificial Sequence Synthetic oligonucleotide
tggaaaattcagtgttatcgctac

SEQ ID NO: 478 - DNA, 21 bp, Artificial Sequence
  Description of Artificial Sequence Synthetic oligonucleotide
aagcaattgcagtttccttga

SEQ ID NO: 479 - DNA, 20 bp, Artificial Sequence
  Description of Artificial Sequence Synthetic oligonucleotide
tatcccccaaccttctaccc

SEQ ID NO: 480 - DNA, 26 bp, Artificial Sequence
  Description of Artificial Sequence Synthetic oligonucleotide
aaggataatattacatttggctgtga

SEQ ID NO: 481 - DNA, 22 bp, Artificial Sequence
  Description of Artificial Sequence Synthetic oligonucleotide
ttttctgtttggcttgacttga

SEQ ID NO: 482 - DNA, 21 bp, Artificial Sequence
  Description of Artificial Sequence Synthetic oligonucleotide
acttgtcacaatgtcaccaca

SEQ ID NO: 483 - DNA, 23 bp, Artificial Sequence
  Description of Artificial Sequence Synthetic oligonucleotide
cacttggagtaacaattgccttt

SEQ ID NO: 484 - DNA, 20 bp, Artificial Sequence
  Description of Artificial Sequence Synthetic oligonucleotide
cactgggaaccaggagctaa

SEQ ID NO: 485 - DNA, 27 bp, Artificial Sequence
  Description of Artificial Sequence Synthetic oligonucleotide
catttaatgtttactgtcacatttctt

SEQ ID NO: 486 - DNA, 25 bp, Artificial Sequence
  Description of Artificial Sequence Synthetic oligonucleotide
aatttacaagacatttaacgaatgg

SEQ ID NO: 487 - DNA, 20 bp, Artificial Sequence
  Description of Artificial Sequence Synthetic oligonucleotide
ttcgaggccagagtccttta

SEQ ID NO: 488 - DNA, 22 bp, Artificial Sequence
  Description of Artificial Sequence Synthetic oligonucleotide
tgcagaagaaaaagtcaggatg

SEQ ID NO: 489 - DNA, 22 bp, Artificial Sequence
  Description of Artificial Sequence Synthetic oligonucleotide
tgcttgctctgataggaaaatg

SEQ ID NO: 490 - DNA, 20 bp, Artificial Sequence
  Description of Artificial Sequence Synthetic oligonucleotide
agcatctcagggccaaaaat

SEQ ID NO: 491 - DNA, 26 bp, Artificial Sequence
  Description of Artificial Sequence Synthetic oligonucleotide
gtaggttgtttttcagtctttattca

SEQ ID NO: 492 - DNA, 22 bp, Artificial Sequence
  Description of Artificial Sequence Synthetic oligonucleotide
cggtaaaataaacaccaagacg

SEQ ID NO: 493 - DNA, 23 bp, Artificial Sequence
  Description of Artificial Sequence Synthetic oligonucleotide
ttgtgttttcaatgagtgtgaag

SEQ ID NO: 494 - DNA, 23 bp, Artificial Sequence
  Description of Artificial Sequence Synthetic oligonucleotide
tgttcttttggatagcatgaagc

SEQ ID NO: 495 - DNA, 23 bp, Artificial Sequence
  Description of Artificial Sequence Synthetic oligonucleotide
gctttcttgtaaagtgtgatggg

SEQ ID NO: 496 - DNA, 22 bp, Artificial Sequence
  Description of Artificial Sequence Synthetic oligonucleotide
ccacacaagtgttctttggttc

SEQ ID NO: 497 - DNA, 45 bp, Artificial Sequence
  Description of Artificial Sequence Synthetic oligonucleotide
gcctccctcgcgccatcagacgcttcttaagcgtcgatggaggag

SEQ ID NO: 498 - DNA, 47 bp, Artificial Sequence
  Description of Artificial Sequence Synthetic oligonucleotide
gccttgccagcccgctcagacgctttgaaacccaaggtacatttcag

SEQ ID NO: 499 - DNA, 45 bp, Artificial Sequence
  Description of Artificial Sequence Synthetic oligonucleotide
gcctccctcgcgccatcagacgctcgtcatctttggagcaggaac

SEQ ID NO: 500 - DNA, 45 bp, Artificial Sequence
  Description of Artificial Sequence Synthetic oligonucleotide
gccttgccagcccgctcagacgctatgcatggcattagcaaagac

SEQ ID NO: 501 - DNA, 51 bp, Artificial Sequence
  Description of Artificial Sequence Synthetic oligonucleotide
gcctccctcgcgccatcagacgcttggtgtagtggaaactaggaattacat

SEQ ID NO: 502 - DNA, 45 bp, Artificial Sequence
  Description of Artificial Sequence Synthetic oligonucleotide
gccttgccagcccgctcagacgcttggattaagaagcaatgccct

SEQ ID NO: 503 - DNA, 48 bp, Artificial Sequence
  Description of Artificial Sequence Synthetic oligonucleotide
gcctccctcgcgccatcagacgcttcagttgcctgaagagaaacataa

SEQ ID NO: 504 - DNA, 45 bp, Artificial Sequence
  Description of Artificial Sequence Synthetic oligonucleotide
gccttgccagcccgctcagacgcttaacagtctgcatggagcagg

SEQ ID NO: 505 - DNA, 46 bp, Artificial Sequence
  Description of Artificial Sequence Synthetic oligonucleotide
gcctccctcgcgccatcagacgctgaacaaaccaggattctagccc

SEQ ID NO: 506 - DNA, 45 bp, Artificial Sequence
  Description of Artificial Sequence Synthetic oligonucleotide
gccttgccagcccgctcagacgctagtggttgccaccttgttacc

SEQ ID NO: 507 - DNA, 42 bp, Artificial Sequence
  Description of Artificial Sequence Synthetic oligonucleotide
gcctccctcgcgccatcagacgctagctctccgcctcccttc

SEQ ID NO: 508 - DNA, 42 bp, Artificial Sequence
  Description of Artificial Sequence Synthetic oligonucleotide
gccttgccagcccgctcagacgctaaggtggctgagggcatc

SEQ ID NO: 509 - DNA, 47 bp, Artificial Sequence
  Description of Artificial Sequence Synthetic oligonucleotide
gcctccctcgcgccatcagacgctgaggaacactggcagttactgtg

SEQ ID NO: 510 - DNA, 50 bp, Artificial Sequence
  Description of Artificial Sequence Synthetic oligonucleotide
gccttgccagcccgctcagacgcttctcttcccaaatctattcctaatcc

SEQ ID NO: 511 - DNA, 44 bp, Artificial Sequence
  Description of Artificial Sequence Synthetic oligonucleotide
gcctccctcgcgccatcagacgctaggacaaagtccggattgaa

SEQ ID NO: 512 - DNA, 46 bp, Artificial Sequence
  Description of Artificial Sequence Synthetic oligonucleotide
gccttgccagcccgctcagacgctcaaagaaacagcaaaatggtga

SEQ ID NO: 513 - DNA, 47 bp, Artificial Sequence
  Description of Artificial Sequence Synthetic oligonucleotide
gcctccctcgcgccatcagacgctttgctccctttacctcttatcaa

SEQ ID NO: 514 - DNA, 45 bp, Artificial Sequence
  Description of Artificial Sequence Synthetic oligonucleotide
gccttgccagcccgctcagacgcttttcacgtcacatacaaacca

SEQ ID NO: 515 - DNA, 47 bp, Artificial Sequence
  Description of Artificial Sequence Synthetic oligonucleotide
gcctccctcgcgccatcagacgctccaagaaaggcttgtgtctacat

SEQ ID NO: 516 - DNA, 48 bp, Artificial Sequence
  Description of Artificial Sequence Synthetic oligonucleotide
gccttgccagcccgctcagacgcttcatccatatttcacattccctaa

SEQ ID NO: 517 - DNA, 48 bp, Artificial Sequence
  Description of Artificial Sequence Synthetic oligonucleotide
gcctccctcgcgccatcagacgcttgtgagtttttgaaatctctgtga

SEQ ID NO: 518 - DNA, 51 bp, Artificial Sequence
  Description of Artificial Sequence Synthetic oligonucleotide
gccttgccagcccgctcagacgcttggtaggtagaaaagagatatttttgg

SEQ ID NO: 519 - DNA, 44 bp, Artificial Sequence
  Description of Artificial Sequence Synthetic oligonucleotide
gcctccctcgcgccatcagacgctgaagcttctgggttttgcac

SEQ ID NO: 520 - DNA, 44 bp, Artificial Sequence
  Description of Artificial Sequence Synthetic oligonucleotide
gccttgccagcccgctcagacgctccaaaagaaagcggttcaag

SEQ ID NO: 521 - DNA, 50 bp, Artificial Sequence
  Description of Artificial Sequence Synthetic oligonucleotide
gcctccctcgcgccatcagacgcttcttatcttccaatgacttcattttt

SEQ ID NO: 522 - DNA, 51 bp, Artificial Sequence
  Description of Artificial Sequence Synthetic oligonucleotide
gccttgccagcccgctcagacgctgcagaaaaataaagatacatacttggt

SEQ ID NO: 523 - DNA, 48 bp, Artificial Sequence
  Description of Artificial Sequence Synthetic oligonucleotide
gcctccctcgcgccatcagacgcttggaaaattcagtgttatcgctac

SEQ ID NO: 524 - DNA, 45 bp, Artificial Sequence
  Description of Artificial Sequence Synthetic oligonucleotide
gccttgccagcccgctcagacgctaagcaattgcagtttccttga

SEQ ID NO: 525 - DNA, 44 bp, Artificial Sequence
  Description of Artificial Sequence Synthetic oligonucleotide
gcctccctcgcgccatcagacgcttatcccccaaccttctaccc

SEQ ID NO: 526 - DNA, 50 bp, Artificial Sequence
  Description of Artificial Sequence Synthetic oligonucleotide
gccttgccagcccgctcagacgctaaggataatattacatttggctgtga

SEQ ID NO: 527 - DNA, 46 bp, Artificial Sequence
  Description of Artificial Sequence Synthetic oligonucleotide
gcctccctcgcgccatcagacgctttttctgtttggcttgacttga

SEQ ID NO: 528 - DNA, 45 bp, Artificial Sequence
  Description of Artificial Sequence Synthetic oligonucleotide
gccttgccagcccgctcagacgctacttgtcacaatgtcaccaca

SEQ ID NO: 529 - DNA, 47 bp, Artificial Sequence
  Description of Artificial Sequence Synthetic oligonucleotide
gcctccctcgcgccatcagacgctcacttggagtaacaattgccttt

SEQ ID NO: 530 - DNA, 44 bp, Artificial Sequence
  Description of Artificial Sequence Synthetic oligonucleotide
gccttgccagcccgctcagacgctcactgggaaccaggagctaa

SEQ ID NO: 531 - DNA, 51 bp, Artificial Sequence
  Description of Artificial Sequence Synthetic oligonucleotide
gcctccctcgcgccatcagacgctcatttaatgtttactgtcacatttctt

SEQ ID NO: 532 - DNA, 49 bp, Artificial Sequence
  Description of Artificial Sequence Synthetic oligonucleotide
gccttgccagcccgctcagacgctaatttacaagacatttaacgaatgg

SEQ ID NO: 533 - DNA, 44 bp, Artificial Sequence
  Description of Artificial Sequence Synthetic oligonucleotide
gcctccctcgcgccatcagacgctttcgaggccagagtccttta

SEQ ID NO: 534 - DNA, 46 bp, Artificial Sequence
  Description of Artificial Sequence Synthetic oligonucleotide
gccttgccagcccgctcagacgcttgcagaagaaaaagtcaggatg

SEQ ID NO: 535 - DNA, 46 bp, Artificial Sequence
  Description of Artificial Sequence Synthetic oligonucleotide
gcctccctcgcgccatcagacgcttgcttgctctgataggaaaatg

SEQ ID NO: 536 - DNA, 44 bp, Artificial Sequence
  Description of Artificial Sequence Synthetic oligonucleotide
gccttgccagcccgctcagacgctagcatctcagggccaaaaat

SEQ ID NO: 537 - DNA, 50 bp, Artificial Sequence
  Description of Artificial Sequence Synthetic oligonucleotide
gcctccctcgcgccatcagacgctgtaggttgtttttcagtctttattca

SEQ ID NO: 538 - DNA, 46 bp, Artificial Sequence
  Description of Artificial Sequence Synthetic oligonucleotide
gccttgccagcccgctcagacgctcggtaaaataaacaccaagacg

SEQ ID NO: 539 - DNA, 47 bp, Artificial Sequence
  Description of Artificial Sequence Synthetic oligonucleotide
gcctccctcgcgccatcagacgctttgtgttttcaatgagtgtgaag

SEQ ID NO: 540 - DNA, 47 bp, Artificial Sequence
  Description of Artificial Sequence Synthetic oligonucleotide
gccttgccagcccgctcagacgcttgttcttttggatagcatgaagc

SEQ ID NO: 541 - DNA, 47 bp, Artificial Sequence
  Description of Artificial Sequence Synthetic oligonucleotide
gcctccctcgcgccatcagacgctgctttcttgtaaagtgtgatggg

SEQ ID NO: 542 - DNA, 46 bp, Artificial Sequence
  Description of Artificial Sequence Synthetic oligonucleotide
gccttgccagcccgctcagacgctccacacaagtgttctttggttc

SEQ ID NO: 543 - DNA, 45 bp, Artificial Sequence
  Description of Artificial Sequence Synthetic oligonucleotide
gcctccctcgcgccatcagcacgatcttaagcgtcgatggaggag

SEQ ID NO: 544 - DNA, 47 bp, Artificial Sequence
  Description of Artificial Sequence Synthetic oligonucleotide
gccttgccagcccgctcagcacgattgaaacccaaggtacatttcag

SEQ ID NO: 545 - DNA, 45 bp, Artificial Sequence
  Description of Artificial Sequence Synthetic oligonucleotide
gcctccctcgcgccatcagcacgacgtcatctttggagcaggaac

SEQ ID NO: 546 - DNA, 45 bp, Artificial Sequence
  Description of Artificial Sequence Synthetic oligonucleotide
gccttgccagcccgctcagcacgaatgcatggcattagcaaagac

SEQ ID NO: 547 - DNA, 51 bp, Artificial Sequence
  Description of Artificial Sequence Synthetic oligonucleotide
gcctccctcgcgccatcagcacgatggtgtagtggaaactaggaattacat

SEQ ID NO: 548 - DNA, 45 bp, Artificial Sequence
  Description of Artificial Sequence Synthetic oligonucleotide
gccttgccagcccgctcagcacgatggattaagaagcaatgccct

SEQ ID NO: 549 - DNA, 48 bp, Artificial Sequence
  Description of Artificial Sequence Synthetic oligonucleotide
gcctccctcgcgccatcagcacgatcagttgcctgaagagaaacataa

SEQ ID NO: 550 - DNA, 45 bp, Artificial Sequence
  Description of Artificial Sequence Synthetic oligonucleotide
gccttgccagcccgctcagcacgataacagtctgcatggagcagg

SEQ ID NO: 551 - DNA, 46 bp, Artificial Sequence
  Description of Artificial Sequence Synthetic oligonucleotide
gcctccctcgcgccatcagcacgagaacaaaccaggattctagccc

SEQ ID NO: 552 - DNA, 45 bp, Artificial Sequence
  Description of Artificial Sequence Synthetic oligonucleotide
gccttgccagcccgctcagcacgaagtggttgccaccttgttacc

SEQ ID NO: 553 - DNA, 42 bp, Artificial Sequence
  Description of Artificial Sequence Synthetic oligonucleotide
gcctccctcgcgccatcagcacgaagctctccgcctcccttc

SEQ ID NO: 554 - DNA, 42 bp, Artificial Sequence
  Description of Artificial Sequence Synthetic oligonucleotide
gccttgccagcccgctcagcacgaaaggtggctgagggcatc

SEQ ID NO: 555 - DNA, 47 bp, Artificial Sequence
  Description of Artificial Sequence Synthetic oligonucleotide
gcctccctcgcgccatcagcacgagaggaacactggcagttactgtg

SEQ ID NO: 556 - DNA, 50 bp, Artificial Sequence
  Description of Artificial Sequence Synthetic oligonucleotide
gccttgccagcccgctcagcacgatctcttcccaaatctattcctaatcc

SEQ ID NO: 557 - DNA, 44 bp, Artificial Sequence
  Description of Artificial Sequence Synthetic oligonucleotide
gcctccctcgcgccatcagcacgaaggacaaagtccggattgaa

SEQ ID NO: 558 - DNA, 46 bp, Artificial Sequence
  Description of Artificial Sequence Synthetic oligonucleotide
gccttgccagcccgctcagcacgacaaagaaacagcaaaatggtga

SEQ ID NO: 559 - DNA, 47 bp, Artificial Sequence
  Description of Artificial Sequence Synthetic oligonucleotide
gcctccctcgcgccatcagcacgattgctccctttacctcttatcaa

SEQ ID NO: 560 - DNA, 45 bp, Artificial Sequence
  Description of Artificial Sequence Synthetic oligonucleotide
gccttgccagcccgctcagcacgatttcacgtcacatacaaacca

SEQ ID NO: 561 - DNA, 47 bp, Artificial Sequence
  Description of Artificial Sequence Synthetic oligonucleotide
gcctccctcgcgccatcagcacgaccaagaaaggcttgtgtctacat

SEQ ID NO: 562 - DNA, 48 bp, Artificial Sequence
  Description of Artificial Sequence Synthetic oligonucleotide
gccttgccagcccgctcagcacgatcatccatatttcacattccctaa

SEQ ID NO: 563 - DNA, 48 bp, Artificial Sequence
  Description of Artificial Sequence Synthetic oligonucleotide
gcctccctcgcgccatcagcacgatgtgagtttttgaaatctctgtga

SEQ ID NO: 564 - DNA, 51 bp, Artificial Sequence
  Description of Artificial Sequence Synthetic oligonucleotide
gccttgccagcccgctcagcacgatggtaggtagaaaagagatatttttgg

SEQ ID NO: 565 - DNA, 44 bp, Artificial Sequence
  Description of Artificial Sequence Synthetic oligonucleotide
gcctccctcgcgccatcagcacgagaagcttctgggttttgcac

SEQ ID NO: 566 - DNA, 44 bp, Artificial Sequence
  Description of Artificial Sequence Synthetic oligonucleotide
gccttgccagcccgctcagcacgaccaaaagaaagcggttcaag

SEQ ID NO: 567 - DNA, 50 bp, Artificial Sequence
  Description of Artificial Sequence Synthetic oligonucleotide
gcctccctcgcgccatcagcacgatcttatcttccaatgacttcattttt

SEQ ID NO: 568 - DNA, 51 bp, Artificial Sequence
  Description of Artificial Sequence Synthetic oligonucleotide
gccttgccagcccgctcagcacgagcagaaaaataaagatacatacttggt

SEQ ID NO: 569 - DNA, 48 bp, Artificial Sequence
  Description of Artificial Sequence Synthetic oligonucleotide
gcctccctcgcgccatcagcacgatggaaaattcagtgttatcgctac

SEQ ID NO: 570 - DNA, 45 bp, Artificial Sequence
  Description of Artificial Sequence Synthetic oligonucleotide
gccttgccagcccgctcagcacgaaagcaattgcagtttccttga

SEQ ID NO: 571 - DNA, 44 bp, Artificial Sequence
  Description of Artificial Sequence Synthetic oligonucleotide
gcctccctcgcgccatcagcacgatatcccccaaccttctaccc

SEQ ID NO: 572 - DNA, 50 bp, Artificial Sequence
  Description of Artificial Sequence Synthetic oligonucleotide
gccttgccagcccgctcagcacgaaaggataatattacatttggctgtga

SEQ ID NO: 573 - DNA, 46 bp, Artificial Sequence
  Description of Artificial Sequence Synthetic oligonucleotide
gcctccctcgcgccatcagcacgattttctgtttggcttgacttga

SEQ ID NO: 574 - DNA, 45 bp, Artificial Sequence
  Description of Artificial Sequence Synthetic oligonucleotide
gccttgccagcccgctcagcacgaacttgtcacaatgtcaccaca

SEQ ID NO: 575 - DNA, 47 bp, Artificial Sequence
  Description of Artificial Sequence Synthetic oligonucleotide
gcctccctcgcgccatcagcacgacacttggagtaacaattgccttt

SEQ ID NO: 576 - DNA, 44 bp, Artificial Sequence
  Description of Artificial Sequence Synthetic oligonucleotide
gccttgccagcccgctcagcacgacactgggaaccaggagctaa

SEQ ID NO: 577 - DNA, 51 bp, Artificial Sequence
  Description of Artificial Sequence Synthetic oligonucleotide
gcctccctcgcgccatcagcacgacatttaatgtttactgtcacatttctt

SEQ ID NO: 578 - DNA, 49 bp, Artificial Sequence
  Description of Artificial Sequence Synthetic oligonucleotide
gccttgccagcccgctcagcacgaaatttacaagacatttaacgaatgg

SEQ ID NO: 579 - DNA, 44 bp, Artificial Sequence
  Description of Artificial Sequence Synthetic oligonucleotide
gcctccctcgcgccatcagcacgattcgaggccagagtccttta

SEQ ID NO: 580 - DNA, 46 bp, Artificial Sequence
  Description of Artificial Sequence Synthetic oligonucleotide
gccttgccagcccgctcagcacgatgcagaagaaaaagtcaggatg

SEQ ID NO: 581 - DNA, 46 bp, Artificial Sequence
  Description of Artificial Sequence Synthetic oligonucleotide
gcctccctcgcgccatcagcacgatgcttgctctgataggaaaatg

SEQ ID NO: 582 - DNA, 44 bp, Artificial Sequence
  Description of Artificial Sequence Synthetic oligonucleotide
gccttgccagcccgctcagcacgaagcatctcagggccaaaaat

SEQ ID NO: 583 - DNA, 50 bp, Artificial Sequence
  Description of Artificial Sequence Synthetic oligonucleotide
gcctccctcgcgccatcagcacgagtaggttgtttttcagtctttattca

SEQ ID NO: 584 - DNA, 46 bp, Artificial Sequence
  Description of Artificial Sequence Synthetic oligonucleotide
gccttgccagcccgctcagcacgacggtaaaataaacaccaagacg

SEQ ID NO: 585 - DNA, 47 bp, Artificial Sequence
  Description of Artificial Sequence Synthetic oligonucleotide
gcctccctcgcgccatcagcacgattgtgttttcaatgagtgtgaag

SEQ ID NO: 586 - DNA, 47 bp, Artificial Sequence
  Description of Artificial Sequence Synthetic oligonucleotide
gccttgccagcccgctcagcacgatgttcttttggatagcatgaagc

SEQ ID NO: 587 - DNA, 47 bp, Artificial Sequence
  Description of Artificial Sequence Synthetic oligonucleotide
gcctccctcgcgccatcagcacgagctttcttgtaaagtgtgatggg

SEQ ID NO: 588 - DNA, 46 bp, Artificial Sequence
  Description of Artificial Sequence Synthetic oligonucleotide
gccttgccagcccgctcagcacgaccacacaagtgttctttggttc

SEQ ID NO: 589 - DNA, 24 bp, Artificial Sequence
  Description of Artificial Sequence Synthetic oligonucleotide
gcctccctcgcgccatcagacgct

SEQ ID NO: 590 - DNA, 24 bp, Artificial Sequence
  Description of Artificial Sequence Synthetic oligonucleotide
gccttgccagcccgctcagacgct

SEQ ID NO: 591 - DNA, 24 bp, Artificial Sequence
  Description of Artificial Sequence Synthetic oligonucleotide
gcctccctcgcgccatcagcacga

SEQ ID NO: 592 - DNA, 24 bp, Artificial Sequence
  Description of Artificial Sequence Synthetic oligonucleotide
gccttgccagcccgctcagcacga

(19) United States
(12) Patent Application Publication
(10) Pub. No.: US 2009/0246789 A1
(43) Pub. Date: Oct. 1, 2009

(54) Gene Mutation Profiling of CSMD1

(75) Inventors: Phillip Buckhaults, Blythewood, SC (US); Christopher L. Farrell, Winnsboro, SC (US); Paul F. Meeh, Chapin, SC (US); Randal L. Croshaw, Columbia, SC (US)

Correspondence Address: DORITY & MANNING, P.A., POST OFFICE BOX 1449, GREENVILLE, SC, 29602-1449, US

(73) Assignee: University of South Carolina, Columbia, SC (US)

(21) Appl. No.: 12/411,024

(22) Filed: Mar. 25, 2009

Related U.S. Application Data
(60) Provisional application No. 61/070,680, filed on Mar. 25, 2008.

(51) Int. Cl. C12Q 1/68

(52) U.S. Cl. 435/6

(57) ABSTRACT

A method for assessing risk of node-positive colorectal cancer in an individual is described. The method includes detecting mutations in CSMD1 genes in a tumor sample from the individual, the mutations being associated with increased risk of node-positive colorectal cancer.

### CROSS-REFERENCE TO RELATED APPLICATION

The present application is based on and claims priority to U.S. Provisional Application Ser. No. 61/070,680 having a filing date of Mar. 25, 2009, which is incorporated by reference herein.

### GOVERNMENT SUPPORT CLAUSE

The present invention was developed with funding from the National Institutes of Health under award 2P0RR017698-06. Therefore, the government retains certain rights in this invention.

### BACKGROUND

Colorectal cancer (CRC) is the third deadliest cancer in the United States with approximately 50,000 deaths occurring annually. Tumors for CRC are found in the colon and rectum, also known as the large intestine. Tumors originate from the columnar epithelial cells found in the lining of the colonic wall.

To characterize the progression of cancer, the International Union Against Cancer and American Joint Committee on Cancer developed the Tumor/Nodes/Metastases Staging System. This system scores the progression of the tumor and scores the tumor to the stage of development. The system utilizes three categories of measurement. The first measurement describes the size and extent of invasion of the primary tumor (T1, T2, T3 and T4).The second measurement describes the extent of metastases to the regional lymph nodes (N0, N1, and N2). The third measurement describes the extent of metastases to distant organs (M0 and M1) such as liver or lungs. Tumors can also be described by Stage I-IV. Stage I and Stage II tumors are of any size (T1-4), but have no evidence of regional lymph node or distant metastases. Stage III tumors can be of any size (T1-4) and with one or more regional lymph node metastases. Stage IV tumors can be any T, any N, but have metastases to distant organs. The prognosis of five-year survival from colorectal cancer is directly correlated with increased stage. The detection of a stage I tumor has a survival rate of >90%. The survival rates of patient with stage II tumors have a survival rate of 72-85%. Patients who are diagnosed with a stage III tumor have only a 65-83% survival rate. Stage IV patients, where the tumors cells are metastasized to other organs, have a survival of only 8%.

Lymph node involvement of CRC is a powerful prognostic indicator, and is used for post-surgery treatment decisions. A patient with a node negative tumor (Stage I or II) will be treated with surgical removal of the primary tumor, but often is not treated with adjuvant chemotherapy. In contrast, a patient with a node positive tumor (Stage III or IV) will be treated with surgery plus adjuvant chemotherapy regimens such as 5-fluorouracil.

Since node status is a critical piece of information used to make treatment decisions for colorectal cancer patients, accuracy of correctly identifying node positive disease is very crucial to the management of colorectal cancer patients. Detecting tumor cells in the regional lymph nodes is rather straightforward, with a low rate of false-positives. Proving with certainty the absence of tumor cells in the regional lymph nodes is more uncertain, in part because the probability of detecting positive nodes is directly proportional to the number of nodes sampled. Correct staging of CRC shows a connection between the clinical outcome of node-negative CRC patients and the number of regional nodes which were examined. The 3 year survival rate of stage II patients is 14% lower when fewer than eight regional lymph nodes are sampled. This decrease in the survival rate is supported by research studies in which stage II cancer patients with lower node counts experienced survival rates that were comparable to node positive stage III patients. This suggests that a fraction of node-negative stage II colorectal cancer patients who experience relapse are actually misclassified node positive patients, and are inappropriately denied adjuvant chemotherapy. The current staging guidelines recommend that pathologists examine at least twelve lymph nodes in CRC patients. However in 2005, studies showed that only 37% of CRC patients had twelve or more nodes inspected for proper staging. It is therefore of great importance to develop alternative methods for the diagnosis of node status. This can be achieved by the discovery of molecular genetic alterations mechanistically linked to tumor pathology.

### SUMMARY

A method for assessing risk of node-positive colorectal cancer in an individual is described. The method includes detecting mutations in CSMD1 genes in a tumor sample from the individual, the mutations being associated with increased risk of node-positive colorectal cancer.

### BRIEF DESCRIPTION OF THE DRAWINGS

A full and enabling disclosure, including the best mode thereof, directed to one of ordinary skill in the art, is set forth more particularly in the remainder of the specification, which makes reference to the appended figures in which:

FIG. 1 illustrates that the Sequential Probability Ratio Test classifies tumors into Allelic Imbalance (above the top line) and Allelic Balance (below the bottom line) categories. The top and bottom lines denote 95% confidence intervals. Light triangles are node-positive tumors and dark triangles are node-negative tumors. Squares are normal DNA samples.

FIG. 2 illustrates CSMD1 copy number decreases in node negative (triangles) and node positive (squares) colorectal tumors.

FIG. 3 illustrates locations of somatic mutations to the CSMD1 gene. A total of 8 nonsynonymous mutations to CSMD1 have been discovered, six of which occur in SUSHI domains. The arrows with M lettered labels denote the locations of mutations described in the present disclosure. The arrows with S lettered labels denote other mutations.

FIG. 4 illustrates CSMD1 mRNA expression in human and rodent epithelial cells. All values are normalized to DLD1, which was equivalent to background levels.

FIG. 5 illustrates (A) Probability of detecting variant alleles. The binomial probability of detecting a variant allele increases with the number of sequencing reads. A single heterozygous mutation in a mixture of 14 tumors is evident as a variant allele present at 4% of all alleles. Sequencing to a depth of 300 fold or greater results in greater than 95% chance of detecting heterozygous variants five or more times. (B) Depth of Coverage of CSMD1 Exons. 90% of CSMD1 exons were sequenced to a depth of 300 fold or greater. (C) Detection of known and novel CSMD1 germline polymorphisms.

FIG. 6 illustrates the Loss of Copy Number of CSMD1. The copy number difference between the normal and tumor show a greater loss in majority of the node positive tumors. The Exon 2 and Exon 30 show a majority of the positive tumors have less than one copy of CSMD1. (A) Exon 2 has twelve of the fourteen node positive tumors with less than one copy. Exon 30 has thirteen of the fourteen node positive tumors with less than one copy. The summary table has twelve out of fourteen node positive tumors with less than one copy and nine of the eleven node negative tumors with more than one copy (B).

Table I illustrates association of node status with Allelic Imbalance (A), Copy number decrease (B), Somatic Mutation (C) and combination of any two 8p alterations (D). (Fisher's exact p=0.0004).
Table II illustrates a summary of colorectal tumors.
Table III illustrates variant alleles.

### DETAILED DESCRIPTION

Reference now will be made in detail to various embodiments of the disclosure, one or more examples of which are set forth below. Each example is provided by way of explanation of the disclosure, not limitation of the disclosure. In fact, it will be apparent to those skilled in the art that various modifications and variations can be made in the present disclosure without departing from the scope or spirit of the disclosure. For instance, features illustrated or described as part of one embodiment, can be used on another embodiment to yield a still further embodiment. Thus, it is intended that the present disclosure covers such modifications and variations as come within the scope of the appended claims and their equivalents.

The present disclosure is generally directed to methods for assessing risk of node-positive colorectal cancer in an individual. The methods include detecting mutations in CSMD1 genes in a tumor sample from the individual, the mutations being associated with increased risk of node-positive colorectal cancer.

In practice of the present invention, reference can be made to the thesis, Identification and Characterization of a Novel Colorectal Tumor Suppressor Gene, Christopher L. Farrell, Doctoral Thesis, University of South Carolina School of Medicine, 2008, which is incorporated herein by reference in its entirety.

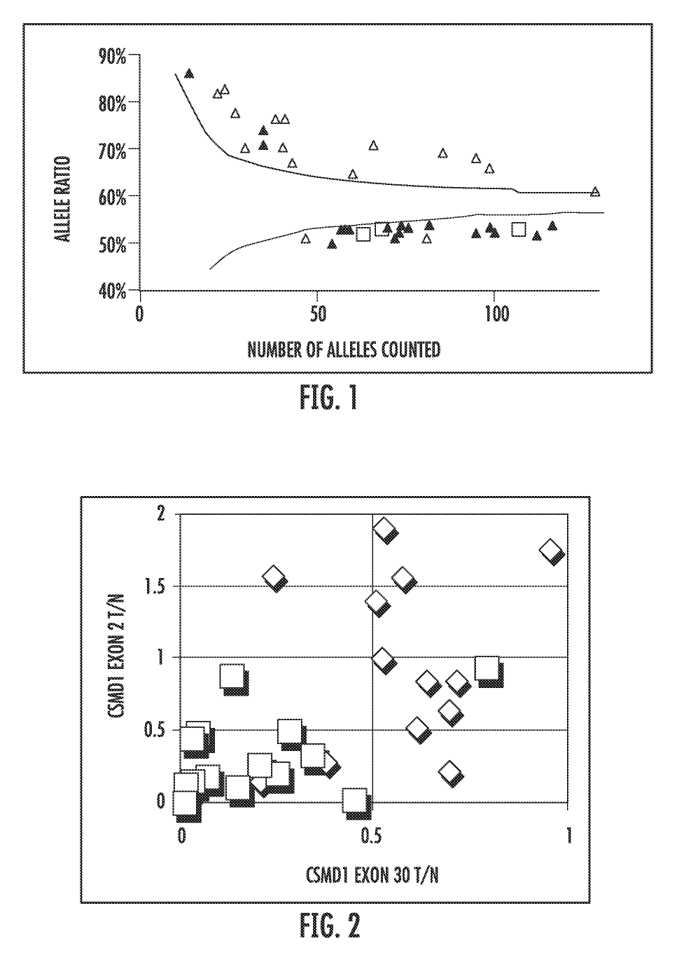

In accordance with the present disclosure, initial characterizations of colorectal tumors focused on identifying genomic deletions in node-positive vs. node-negative colorectal tumors. Epithelium represents a very minor component of tumor and normal tissue specimens. Therefore, laser capture micro dissection was utilized to purify normal and tumor epithelial cells from each of 30 surgical specimens derived from node negative and node positive colorectal cancer patients. Normal and tumor genomic DNA was isolated from these specimens and microsatellite PCR was performed at 8 locations along the p arm of chromosome 8. A digital allele counting approach was used to precisely quantify the ratios of the maternal and paternal alleles, and the resultant allele ratios analyzed by a Sequential Probability Ratio Test as illustrated in FIG. 1. By using this approach, all tumors are classified into two groups, those exhibiting 8p Allelic Imbalance, and those with balanced 8p alleles. Chromosome 8p is frequently imbalanced in node-positive tumors, yet infrequently imbalanced in node-negative tumors (Table I (A)). One allele of an 8p tumor suppressor gene is deleted in node-positive tumors, and that gene sequencing would reveal somatic alterations in the remaining allele of this gene in node-positive tumors. The experiments of the present disclosure suggest that a frequent molecular defect on chromosome 8p is linked with the ability of colorectal cancers to metastasize to the regional lymph nodes.

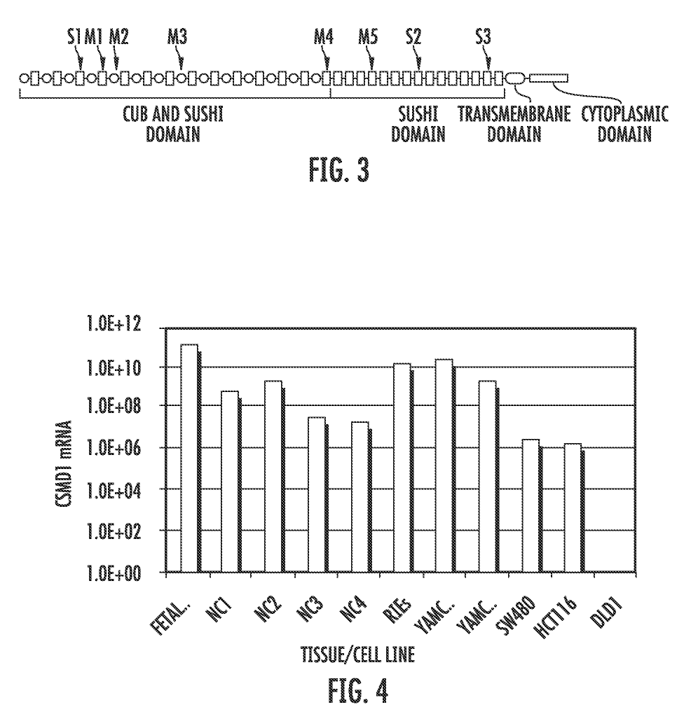

In order to identify candidate tumor suppressor genes, all known genes in breast and colon cancers were sequenced, and identified over 1800 genes with somatic mutations in these two tumor types. Fourteen genes located on 8p were found to be mutated once in a discovery set of 11 colorectal tumors. The CSMD1 gene was the only one of these genes which suffered additional somatic mutations in a validation set of 24 colorectal tumors. In total, three non-synonymous mutations were identified in 34 colorectal cancers, indicating that CSMD1 may be a novel 8p tumor suppressor gene. 8p copy loss is associated with Lymph Node Involvement. All tumors were pre-screened for mismatch repair deficiency by amplifying the BAT26 locus for each tumor—normal pair and checking the sizes of the PCR products by gel electrophoresis. During this procedure, 3 tumors were discovered to be unstable at the BAT26 locus, and these three tumors were removed and not studied further. All tumors used in this disclosure are all mismatch repair proficient, and are therefore presumed to be aneuploid. Allelic imbalance may arise either from allele loss or gain. In order to directly implicate allele loss, as would accompany mutation of a tumor suppressor gene, copy number measurements were performed by quantitative real-time PCR. The total number of genomes were quantitated using realtime PCR against Long Intersperse Nuclear Elements (LINE) and these values were used to normalize for total DNA quantity. The total number of CSMD1 alleles at two locations within the CSMD1 gene (Exon 2 and Exon 30) were quantitated using realtime PCR against these two exons. The number of CSMD1 alleles in the tumor was divided by the number of CSMD1 alleles found in the matched normal sample. These ratios for CSMD1 Exon 2 (tumor/normal) and CSMD1 exon 30 (tumor/normal) for both node negative and node positive tumors are shown in FIG. 2. A cutoff ratio of 0.5 T/N was chosen to identify tumors that had lost at least one CSMD1 allele. The majority of node positive tumors have lost at least one CSMD1 allele, whereas the majority of node negative tumors have retained both CSMD1 alleles. Furthermore, most tumors lose both exons of the CSMD1 gene simultaneously. Combining copy number decreases at both CSMD1 exons was able to detect node positive disease with 86% sensitivity and 82% specificity (Table I (B)). These results are in good agreement with allelic imbalance studies, and support the notion that one or more alleles of CSMD1 are lost in node positive colorectal tumors.

Because of observations that associated 8p allelic imbalance with positive lymph node status, it was hypothesized that CSMD1 could be mutated preferentially in node positive disease. In order to test this hypothesis, the sequence of CSMD1 was determined in an additional panel of 26 early and late colorectal cancers, and identified six nonsynonymous somatic mutations to CSMD1, five which were found in metastatic (node-positive) disease. CSMD1 encodes a large, type I transmembrane protein located on the surfaces of neuronal and epithelial cells. The mature protein has an extracellular N-terminal domain of 3,477 amino acids, a single membrane spanning domain, and a short cytoplasmic tail of 56 amino acids.

The function of the protein is unknown, but it is thought to participate in cell migration, in part because it is localized to the leading edges of neuronal growth cones.

The gene encoding CSMD1 is comprised of 71 exons, and is located on chromosome 8p23.1. This region of chromosome 8 is frequently deleted in squamous cell carcinomas of the head and neck. Deletions to the entire p arm of chromosome 8 are frequently detected in advanced cancers of the breast, colon and prostate. Additionally, decreased expression of CSMD1 is associated with advanced prostate cancer.

A high-throughput approach was developed to gene mutation discovery that is based on massively parallel picotiter plate pyrosequencing. The approach was validated by detecting mutations in the KRAS and BRAF oncogenes, and was used to discover novel mutations in CSMD1.

A variant allele (either germline polymorphism or somatically acquired mutation) derived from a single sample heterozygous for a sequence alteration would be present in an equimolar mixture of genomic DNA from 12-14 tumors at a concentration of 4%. Statistical modeling experiments revealed that if such a locus were sequenced to a depth of greater than or equal to 300-fold coverage, these variants would be detected with five or more independent sequencing reads 95% of the time. CSMD1 amplicons were sequenced to an average depth of 1800 fold, with over 90% of amplicons represented by greater than 300 sequencing reads per sample.

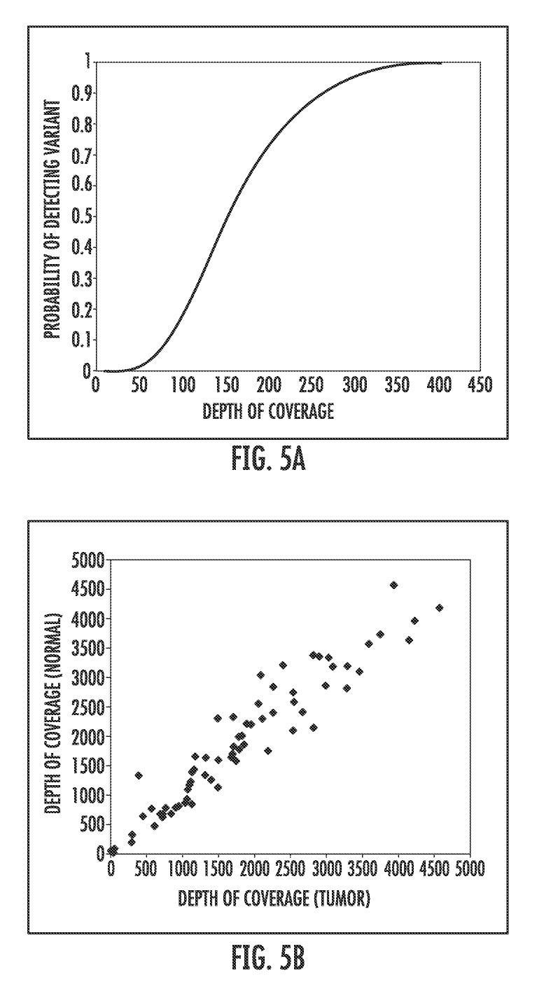

Sequence variants were detected in the tumor samples that were absent in the normal samples. These variants represented candidate somatic mutations, which were independently confirmed by sequencing of the individual tumor and normal specimens using Sanger methodology. In total, the sequence of 11,743 CSMD1 nucleotides was determined from 26 colorectal cancers and matched normal samples, for a total of 1.2 Mb of DNA sequence, and identified six candidate somatic mutations (Sequence Listing and Table I (B)). Sanger sequencing of the mutant loci confirmed that these six alterations were present in five of the 26 colon cancers studied (19%) and that all alterations were absent in corresponding normal DNA samples. Five of the six somatic mutations discovered in CSMD1 are nonsynonymous substitutions, suggesting that these CSMD1 alterations may be functionally significant to the development of colorectal cancers. The overall nonsynonymous mutation rate for CSMD1 was found to be 5.7 mutations per Mb of diploid tumor DNA, which is significantly higher than background rates previously reported for colorectal and most other cancers (Binomial p=3.7×10−4). Two of the mutations introduce premature stop codons prior to the transmembrane domain and are therefore likely to abolish normal function. Two different heterozygous mutations (genomic positions 1595339 and 1687052) were found in the same tumor, which if located on opposing alleles, would indicate total absence of wild-type CSMD1 sequences for that sample. The majority of nonsynonymous mutations discovered to date are found in SUSHI domains, which are highly-conserved in CSMD1 in other species and in SUSHI domains found in other members of the CSMD family of proteins (see FIG. 3). Seven of eight somatic mutations discovered were found in advanced (node-positive) disease, suggesting that CSMD1 mutation may be one mechanism by which colorectal cancers develop the ability to metastasize to the regional lymph nodes.

Somatic mutations to CSMD1, 8p AI, and CSMD1 copy number decreases are three different measures of the same genetic event: loss of CSMD1. In this regard, the sensitivity, specificity, and positive and negative predictive values of each test were examined individually, and in combination. The combined test required that a tumor have two or more of these alterations and have an 86% sensitivity and an 85% specificity (Table I(D)). Two of 13 node-negative tumors were positive for the combined test. It is believed that these two tumors are likely to be mis-diagnosed node positive tumors, and therefore at higher risk for recurrence.

To provide additional justification for determining the potential diagnostic utility of CSMD1 loss and Fibronectin overexpression during colorectal cancer progression, the adhesive, invasive, and migratory properties of colorectal cancer cells engineered to express either CSMD1 or FN1 have been tested. It has been determined that CSMD1 promotes cell adhesion to colorectal cancer cell extracellular matrix, matrigel, and laminin, strengthening the hypothesis that loss of CSMD1 allows cell detachment from the underlying basement membrane. Also, Fibronectin overexpression in colorectal cancer cells leads to increased invasion, migration, and colony formation ability in vitro, strengthening the hypothesis that FN1 overexpression facilitates lymph node involvement by promoting migration and cell survival.

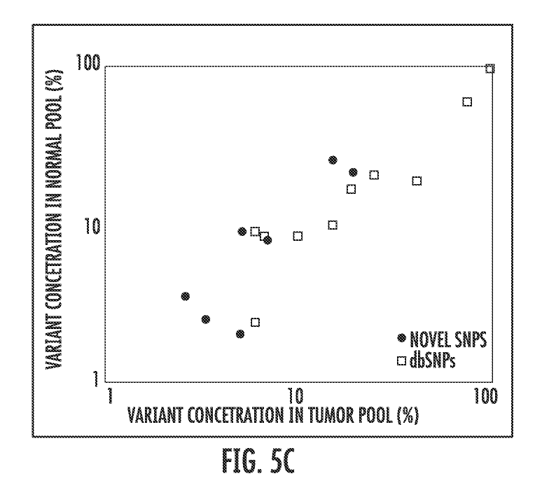

CSMD1 is known to be expressed in migrating neurons; however, its expression in colorectal cancer epithelium has not been studied. To determine if CSMD1 mRNA is expressed in normal and tumor epithelial cells, CSMD1 transcripts were quantitated using quantitative realtime PCR in human and rodent epithelial cells. Young Adult Mouse Colonocytes (YAMCs) transformed with a temperature-sensitive T-antigen and grown at both permissive and non-permissive temperatures, normal Rat Intestinal Epithelial cells (RIEs), human colorectal cancer cell lines SW480, DLD1, and HCT116 plus three normal human colonic mucosae samples derived from surgical specimens were analyzed. Commercially available fetal brain mRNA was utilized as a positive control. CSMD1 expression was normalized to either EEF1A1 (human) or GAPDH (mouse and rat) housekeeping genes. FIG. 4 shows that YAMCs, RIEs, SW480's, and normal human colonic mucosa all express significant levels of CSMD1 mRNA. Notably, DLD1 cells do not express detectable CSMD1 mRNA. These results document that CSMD1 is expressed in normal intestinal epithelium and in some colorectal cancer cell lines and that CSMD1 is not expressed in DLD1 cells, justifying the utilization of DLD1 cells for CSMD1 expression experiments.

Because CSMD1 mutations in the primary tumor correlate with positive lymph node status, this result will have significant impact on the diagnosis of colorectal cancer patients, especially those for whom insufficient numbers of lymph nodes were examined. Biopsy materials taken from the primary tumor (including samples removed during colonoscopy, where no regional lymph nodes are available for examination) can be subjected to deep sequencing of the CSMD1 gene and node-positive tumors identified independent of node sampling depth. Next generation sequencing platforms such as 454, Solexa, and SOLID can drill deep enough to detected mutations in crude clinical samples, without need for tumor cell purification.

The following examples are meant to illustrate the invention described herein and are not intended to limit the scope of this invention.

### EXAMPLES

To further investigate the frequency of CSMD1 mutations in early and late-stage colorectal cancers, the CSMD1 gene was sequenced in a panel of 12 pre-metastatic and 14 metastatic colorectal cancers and five nonsynonymous somatic mutations that are associated with advanced disease were identified.

### Methods

### Statistical Calculations

The cumulative binomial distribution function was evaluated to calculate the probability of observing a 4% variant five or more times as a function of the total number of sequencing reads obtained. Evaluation of this function revealed that with 300 or more sequencing reads per amplicon, the probability of detecting a 4% variant was greater than 0.95.

### Tumor Specimens and DNA Isolation

Twenty-eight frozen paired colorectal normal and tumor samples consisting of 12 lymph-node negative tumors and 16 lymph-node positive tumors were obtained through the South Carolina Cancer Center Tissue Bank. These tissue samples were cut into 10 μm thick slices and fixed onto Sigma silane-prep™ slides by dehydrating in sequential baths of 75%, 95%, and 100% ethanol and xylene. Tumor and normal epithelial cells were microdissected using an Arcturus PixCell© IIe Laser Capture Microdissection microscope (Molecular Devices, Sunnyvale, Calif.). Tumor and normal genomic DNA was purified from microdissected epithelial cells using Qiagen™ QIAamp® DNA Micro Kit (Qiagen, Valencia, Calif.). DNA was quantitated using real-time PCR primers directed against Long Interspersed Nuclear Element sequences (LINEF- AAAGCCGCTCAACTACATGG, LINER- CTCTATTTCCTTCAGTTCTGCTC, Integrated DNA Technologies, Coralville, Iowa). Quantitative real-time PCR was performed using 6.25 μL iTaq supermix (Bio-Rad, Hercules Calif.), 1.25 μL of 2 μM LINEF, 1.25 μL of 2 μM LINER, 2.5 μL of PCR water (Invitrogen, Carlsbad, Calif.) and 1.25 μL of DNA template. Thermal cycling was performed using a MyIQ Thermal Cycler (Bio-Rad, Hercules Calif.) and the following protocol: One cycle, 95° C. for 1 min; 60 cycles of 94° C. for 10 sec, 59° C. for 30 sec; one cycle, 70° C. for 5 min. Mismatch repair proficient tumors were identified by assessing the stability of the BAT26 microsatellite locus. BAT26 microsatellite PCR products were prepared using 6.25 μL iTaq supermix (Bio-Rad, Hercules Calif.), 1.25 μL of 2 μM BAT26F (TGACTACTTTTGACTTCAGCC), 1.25 μL of 2 μM BAT26R (AACCATTCAACATTTTTAACCC), 2.5 μL of PCR water (Invitrogen, Carlsbad, Calif.) and 1.25 μL of DNA template. Thermal cycling was performed MyIQ Thermal Cycler (Bio-Rad, Hercules Calif.) and the following protocol: One cycle, 95° C. for 1 min; 60 cycles of 94° C. for 10 sec, 59° C. for 45 sec; 1 cycle 68° C. for 5 min. PCR products were analyzed by electrophoresis on a 3% agarose gel and photographed using an Alpha Imager and Quantity One™ software (Alpha Innotech, San Leandro, Calif.).

### Whole Genome Amplification of DNA Samples

Tumor and normal genomic DNA templates from microsatellite-stable tumors were amplified using the Qiagen™ REPLI-g Mini Kit. Each Whole Genome Amplification (WGA) reaction was programmed using >10 ng of human genomic DNA (˜3,000 diploid genomes). For each sample, at least 2 independent WGA reactions were performed and the amplified DNA was quantitated using real-time PCR against LINE sequences. Equivalent amounts of WGA DNA from each sample were pooled, re-quantitated, and diluted to a standard concentration of 50 ng/μL.

PCR Amplification of CSMD1, KRAS, & BRAF PCR primers were designed to amplify all exons of the CSMD1, KRAS, and BRAF genes, using PRIMER3 (http://frodo.wi.mit.edu/primer3/input.htm) (Primer Sequence Tables A & B). Forward and reverse primers were tagged with linker sequences A and B as described in the Guide to Amplicon Sequencing, 454 Life Sciences (Branford, Conn.). Preparative PCR was performed using 6.25 μL of iProof supermix (Bio-Rad, Hercules Calif.), 1.25 μL of 2 uM of F primer, 1.25 μL of 2 μM R primer, 2.4 μL of PCR water (Invitrogen, Carlsbad Calif.) 0.1 L of 50×Sybr Green (Invitrogen, Carlsbad Calif.) and 1.25 μL of WGADNA template (>50 ng). Thermal cycling was performed using a MyIQ Thermal Cycler (Bio-Rad, Hercules, Calif.) and the following touchdown protocol: One cycle, 95° C. for 2 min; 3 cycles of 94° C. for 10 sec, 64° C. for 10 sec, 70° C. for 30 sec; 3 cycles of 94° C. for 10 sec, 61° C. for 10 sec, 70° C. for 30 sec; 3 cycles of 94° C. for 10 sec, 58° C. for 10 sec, 70° C. for 30 sec; 50 cycles of 94° C. for 10 sec, 57° C. for 10 sec, 70° C. for 30 sec. The PCR products were analyzed by electrophoresis on a 3% agarose gel and photographed using an Alpha Imager and Quantity One™ software (Alpha Innotech, San Leandro, Calif.). Amplicons were purified using SPRI Ampure beads (Agencourt, Beverly, Mass.) following the manufacturers protocol.

### Sequencing CSMD, KRAS, & BRAF Amplicons

SPRI-purified CSMD1, KRAS, and BRAF amplicons (96) from each sample were pooled together. In order to increase throughput, four superpools of samples were generated as follows, Node Negative Normals (12 patients), Node Negative Tumors (12 patients), Node Positive Normals (14 patients) and Node Positive Tumors (14 patients). Each pool of amplicons were quantitated using the Quanti-iT Picogreen® dsDNA Assay (Invitrogen, Carlsbad, Calif.). The four amplicon pools were subjected to emulsion PCR using emPCR Kits II and III (Roche Diagnostics, Indianapolis, Ind.) and sequenced on the GSFLX genome sequencer (University of South Carolina Environmental Genomics Core Facility, Columbia, S.C.). The sequences were compared to reference sequences and potential somatic mutations identified by using the Amplicon Variant Analysis software (454 Life Sciences, Branford, Conn.).

### Verification of Somatic Mutations

Amplicons containing sequence variants in the tumor pools and absent in both normal pools were independently re-sequenced from separate tumor amplicons using traditional Sanger methodology (University of South Carolina Environmental Genomics Core Facility, Columbia, S.C.). Normal DNA matching tumors containing verified mutations were then sequenced to distinguish somatic mutations from rare germline variants.

### Results

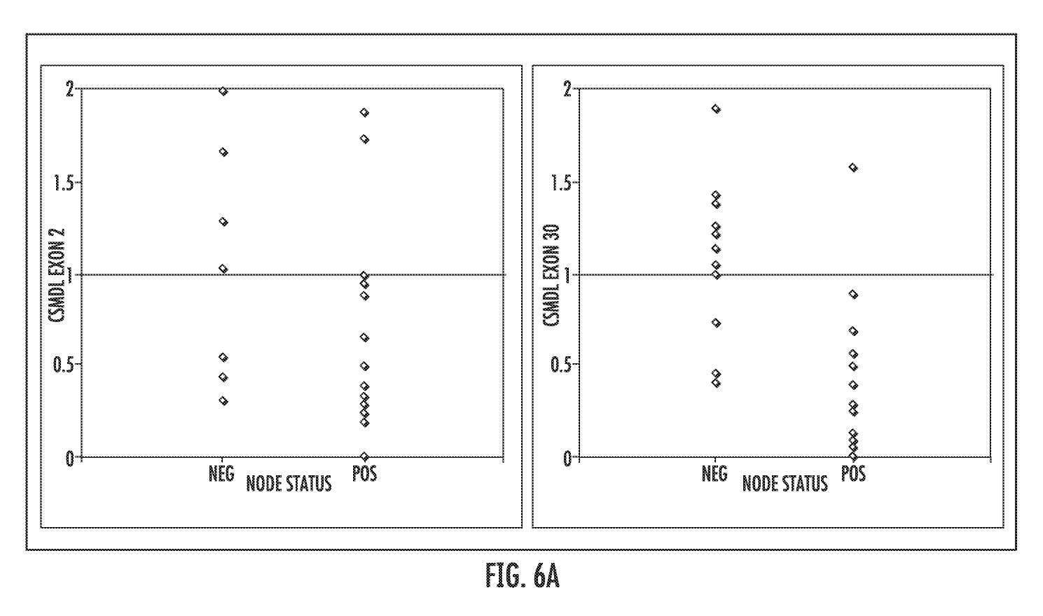

A variant allele (either germline polymorphism or somatically acquired mutation) derived from a single sample heterozygous for the alteration would be present in an equimolar mixture of 12-14 tumor genomes at a concentration of 4%. Binomial probability calculations revealed that if such a locus were sequenced to a depth of greater than or equal to 300-fold coverage, a 4% variant would be detected with five or more independent sequencing reads over 95% of the time (FIG. 5a).

Seventy-three PCR amplicons covering all exons of CSMD1, eighteen amplicons covering all coding exons of BRAF and five amplicons covering all coding exons of the KRAS oncogene were prepared from 26 colorectal cancers and patient-matched normal tissue. These ninety-six PCR amplicons were quantitated and equimolar amounts were pooled together. Four groups of tumor or normal samples were prepared from the patient specimens described in Table II. Samples were grouped according to pathologic stage, as follows: Node negative tumors (12 samples), node negative normals (12 samples), node positive tumors (14 samples) and node positive normals (14 samples). The four groups of amplicons were bidirectionally sequenced with two runs of the 454FLX genome sequencer (454 Life Sciences, Branford, Conn.) with over 500,000 individual sequencing reads obtained.

CSMD1 amplicons were sequenced to an average depth of 1800 fold, with over 90% of amplicons represented by greater than 300 sequencing reads per sample (FIG. 5b). Consistent with the predictions made from statistical modeling experiments, the presence of 17 sequence variants that were present at similar concentrations in both tumor and normal pools were noted (FIG. 5c). Ten of these variants are previously-described single nucleotide polymorphisms (SNPs) in the KRAS, BRAF and CSMD1 genes while seven variants correspond to previously unreported SNPs in the CSMD1 gene (Table III (A)). The vast majority of these germline polymorphisms are synonymous substitutions, resulting in no change in the amino acid sequence, indicating this gene to be under positive selective pressure to maintain wild-type sequence during human development.

In addition to detecting germline polymorphisms, a number of sequence variants in the tumor samples that were absent in the normal samples were detected. These variants represented candidate somatic mutations, which were independently confirmed by sequencing of the individual tumor and normal specimens using Sanger methodology. Seven different candidate somatic mutations in the KRAS oncogene were detected (Table III (B)). Sanger sequencing of the mutant loci revealed these seven KRAS alterations were present in 9 of the 26 colon cancers studied (35%). In addition, four different candidate somatic mutations in the BRAF oncogene were detected. Sanger sequencing of the mutant loci detected one of the four BRAF mutations (Table III (B)), while three of the mutations were undetectable by this method. The frequency and locations of these mutations are similar to observations made over a large number of colorectal tumors (http://www.sanger.ac.uk/genetics/CGP/cosmic/).

The sequence of 11,743 CSMD1 nucleotides from 26 colorectal cancers and matched normal samples was determined, for a total of 1.2 Mb of DNA sequence, and identified six candidate somatic mutations (Table III (B)). Sanger sequencing of the mutant loci confirmed that these six alterations were present in five of the 26 colon cancers studied (19%) and that these alterations were absent in corresponding normal DNA samples.

Five of the six somatic mutations discovered are nonsynonymous substitutions, indicating that these CSMD1 alterations are functionally significant to the development of colorectal cancers. The background rate of nonsynonymous mutations in colorectal cancers has been estimated to be between 0.55-2.35 mutations per Mb [10]. The overall nonsynonymous mutation rate for CSMD1 was found to be 8.2 mutations/Mb (5 mutations per 0.61 Mb of diploid tumor DNA), which is significantly higher than even the highest estimates of background rate of nonsynonymous mutations. (Binomial p=3.4×10^{—}) [9-12].

### Discussion

Cancer genome sequencing efforts have shown that the vast majority of cancer genes are mutated at frequencies less than ten percent of tumors. Therefore, gene sequencing strategies that are easily scalable to large numbers of tumor samples are needed to accurately determine mutation frequencies or to correlate mutations to early or late-stage disease. A high-throughput mutation discovery strategy based on massively parallel picotiter plate pyrosequencing was developed that allows investigators to quickly interrogate over 1 Mb DNA sequence and identify variant alleles present at low concentrations in mixtures of clinical samples. This strategy has been used to determine an association between somatic mutations in CSMD1 and metastatic progression of colorectal cancer.

The data demonstrate that CSMD1 is an important 8p tumor suppressor gene. This idea is supported by several lines of evidence. First, two of the mutations introduce premature stop codons prior to the transmembrane domain and therefore abolish normal function. Second, two different heterozygous mutations (genomic positions 1595339 and 1687052) were found in the same tumor, which if located on opposing alleles, would indicate total absence of wild-type CSMD1 sequences for that sample. Third, the majority of nonsynonymous mutations discovered to date are found in SUSHI domains, which are highly-conserved in CSMD1 in other species and in SUSHI domains found in other members of the CSMD family of proteins (FIG. 3). Finally, five of the six somatic mutations observed occurred in advanced (node-positive) disease, indicating that CSMD1 mutation is one mechanism by which colorectal cancers develop the ability to metastasize to the regional lymph nodes.

As demonstrated, CSMD1 mutations play a role in the development of colorectal cancer. Loss of function mutations along with loss of heterozygosis' compromises cell attachments mediated by CSMD1, leading to decreased cell adhesion, invasion or metastasis. Associations between CSMD1 mutation and poor clinical outcome its role in normal and tumor epithelial cell adhesion and migration are indicated.

### Copy Number Data for CSMD1 in Colorectal Tumors

Previous data of the colorectal panel demonstrated that a majority of the node positive colorectal tumors showed increased loss of heterozygosity and allelic imbalance on 8p22. The copy analysis of the CSMD1 was used to establish the CIN of colorectal tumors at the 8p23.1 locus where the CSMD1 gene is located. To examine copy number changes in 8p23.1, two locations, Exon 2 and Exon 30 of the CSMD1 gene were examined.

The quantification of CSMD1 copy number in the colorectal panel was performed using real-time PCR for both the LINE primers and two sets of primers in the exons of the CSMD1 gene. The LINE primers, consisted of a LINE (F) Forward-AAAGCCGCTCAACTACATGG and a LINE (R) Reverse-CTCTATTTCCTTCAGTTCTGCTC, were used for each tumor sample and corresponding normal sample to quantify the amount of DNA in the sample. The CSMD1 primers were used to determine the number of copies of the CSMD1 gene. The CSMD1 primers for real-time PCR included Exon 2 forward- GCCTCCCTCGCGCCATCAG ACGCTTTTTGTGTGTGTTTTCTCTTCCA and Exon 2 reverse-GCCTTGCCAGCCCGCTCAGACGCTTCATAATCTGTGTATTCAAA CAGTGC, and Exon 30 forward- GCCTCCCTCGCGCCATCAGACGCTGCCGTA TGACATAATTACTATTCTTTT and Exon 30 reverse- GCCTTGCCAGCCCGCTCA GACGCTCCCACTCAA ATTCAGGCAAT.

The average cycle threshold (Ct) values of the LINE PCR products from each sample were compared to the average Ct values of the CSMD1 PCR products, shown in the following equations:

AVG of Normal CSMD1 Exon−AVG of Normal LINE=Normalized Normal CSMD1 Copies

AVG of Tumor CSMD1 Exon−AVG of Tumor LINE=Normalized Tumor CSMD1 Copies

The ratio of tumor CSMD1 alleles to normal CSMD1 alleles can be calculated from the real time data as following:

$$\frac{Normalized Tumor CSMD 1 Copies}{Normalized Normal CSMD 1 Copies} = Ratio of CSMD 1 Copies$$

The copy number difference between tumor and normal showed if the tumor DNA sample was retained in both copies of the CSMD1 gene or the loss of copy of the CSMD1 gene.

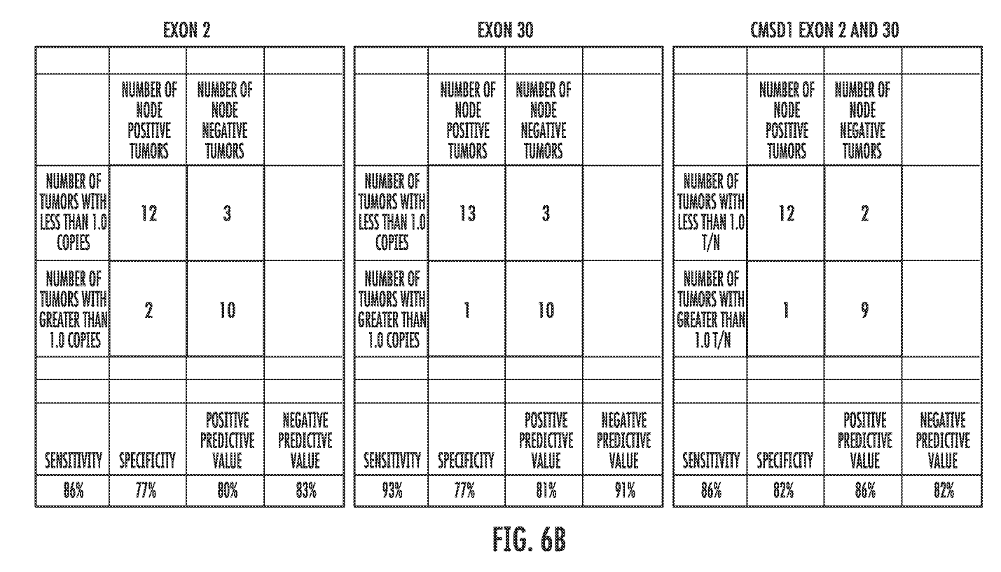

Majority of the node negative tumors showed a copy number retention of both CSMD1 genes in Exon 2 and Exon 30. The two CSMD1 gene copies were observed in ten out of the thirteen samples at the Exon 2 and Exon 30 loci (FIG. 6A). The majority of the node negative tumors had a difference of value of one or higher. The one node negative tumor with a non-synonymous mutation had a lower copy number in Exon 2 and Exon 30 loci, which suggests copy number loss. The node positive tumors showed loss of one of the two CSMD1 copies in both Exon 2 and Exon 30. The Exon 2 locus had twelve of the fourteen tumors with loss of copy number (FIG. 6B). The other locus, Exon 30, had similar results with thirteen out of the fourteen tumors with copy number loss (FIG. 6B). The results are reflective of the data presented herein which shows chromosomal instability across the 8p arm. The copy number data suggests that the majority of the node positive tumors have CSMD1 loss at Exon 2 and 30.

In three of the node positive tumors (3357, 11057, 29271, see Table II), the results showed the possibility of a homozygous deletion in one of the two exons of CSMD1 gene, previous sequence data has observed this even. The ratios of the 3357 and 29271 at the Exon 2 site were below 0.1, which suggest that these two tumors have a deletion around this locus. The ratios of 11057 and 29271 were also below 0.1 in Exon 30, which showed possible homozygous deletions.

Allelic Imbalance of CSMD1 with 454 Sequencing

The SPRI-Ampure bead purified PCR products (CSMD1, KRAS, and BRAF exons), which consisted of 96 amplicons for each sample, are pooled together. These PCR pools for each sample are to remain separate through 454 primer tagging or physical means (454 gasket −2 region, 4 region & 16 region).

The amplicon pools are quantified using the Quanti-iT Picogreen® dsDNA Assay (Invitrogen, Carlsbad, Calif.) and Fluoroskan Ascent FL (Thermo Fisher Scientific Inc., Waltham, Mass.). The amplicon pools for each sample are amplified by the emulsion PCR method using the emPCR Kite II and III (Roche Diagnostics, Indianapolis, Ind.) and sequenced on the GSFLX genome sequencer (University of South Carolina Environmental Genomics Core Facility, Columbia, S.C.). The 454 sequences were compared to the reference sequences from NCBI and identified nucleotide changes with the Amplicon Variant Analysis software (454 Life Sciences, Branford, Conn.).

Using the known SNP variants (example: table III), a SNP ratio can be determined for each tumor and then be plotted on the Sequential Probability Ratio Test for statistical significance. The test states the hypothesis that if the laser capture microdissection tumor samples contain up to 50% normal and 50% tumor DNA, the alleles would be distributed with two alleles from normal DNA and one allele from the tumor DNA which would give the observed loss of heterozygosity allele ratio of at least 67%. This means that if the tumor shows allelic imbalance (AI), then the percentage will be greater than 50%. The equation was represented for the upper boundary to show the number alleles required to statistically be AI:

$$Ratio = \frac{LN 16}{( N * LN 2 )} + \frac{LN 1.5}{LN 2} ( \begin{matrix}where N is the number \\ of allele counts\end{matrix} )$$

The lower boundary was represented for the number of allele to show the number of alleles required for balance, which is a ratio of 50%.

In the interest of brevity and conciseness, any ranges of values set forth in this specification are to be construed as written description support for claims reciting any sub-ranges having endpoints which are whole number values within the specified range in question. By way of a hypothetical illustrative example, a disclosure in this specification of a range of 1-5 shall be considered to support claims to any of the following sub-ranges: 1-4; 1-3; 1-2; 2-5; 2-4; 2-3; 3-5; 3-4; and 4-5.

These and other modifications and variations to the present disclosure can be practiced by those of ordinary skill in the art, without departing from the spirit and scope of the present disclosure, which is more particularly set forth in the appended claims. In addition, it should be understood that aspects of the various embodiments can be interchanged both in whole or in part. Furthermore, those of ordinary skill in the art will appreciate that the foregoing description is by way of example only, and is not intended to limit the disclosure so as further described in such appended claims.

TABLE I
ALLELIC IMBALANCE TEST

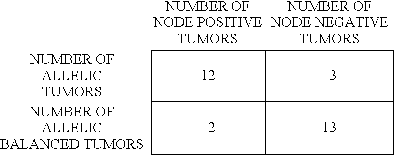

| | | POSITIVE | POSITIVE |
|---|---|---|---|
| | | PREDICTIVE | PREDICTIVE |
| SENSITIVITY | SPECIFICITY* | VALUE | VALUE |
| 86% | 81% | 80% | 87% |

CSMD1 COPY NUMBER DECREASE TEST

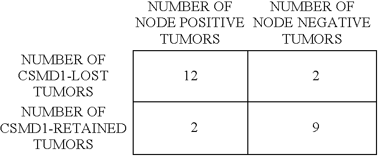

| | | POSITIVE | POSITIVE |
|---|---|---|---|
| | | PREDICTIVE | PREDICTIVE |
| SENSITIVITY | SPECIFICITY* | VALUE | VALUE |
| 86% | 82% | 86% | 82% |

CSMD1 SOMATIC MUTATION TEST

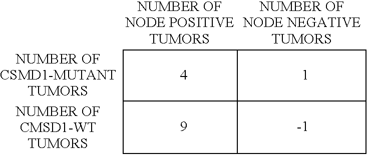

| | | POSITIVE | POSITIVE |
|---|---|---|---|
| | | PREDICTIVE | PREDICTIVE |
| SENSITIVITY | SPECIFICITY | VALUE | VALUE |
| 31% | 92% | 80% | 55% |

COMBINED 8p DELETION TEST*

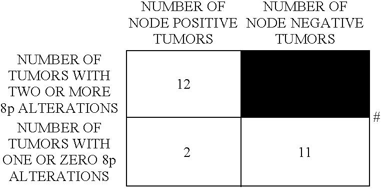

| | | POSITIVE | POSITIVE |
|---|---|---|---|
| | | PREDICTIVE | PREDICTIVE |
| SENSITIVITY | SPECIFICITY* | VALUE | VALUE |
| 86% | 85% | 86% | 85% |

TABLE II

| | | | | | | | | | BAT26 |
|---|---|---|---|---|---|---|---|---|---|
| PATIENT | AGE | SEX | RACE | T | N | M | STAGE | HISTOLOGY | LOCUS |
| 12197 | 73 | F | AA | 1 | 0 | 0 | I | ADENOCARCINOMA | STABLE |
| 11330 | 72 | F | AA | 2 | 0 | 0 | I | ADENOCARCINOMA | STABLE |
| 23662 | 63 | M | CA | 2 | 0 | 0 | I | ADENOCARCINOMA | STABLE |
| 30232 | 74 | M | CA | 2 | 0 | 0 | I | ADENOCARCINOMA | STABLE |
| 10236 | 71 | F | NA | 3 | 0 | 0 | II | ADENOCARCINOMA | STABLE |
| 12304 | 45 | M | AA | 3 | 0 | 0 | II | ADENOCARCINOMA | STABLE |
| 15095 | 67 | M | NA | 3 | 0 | 0 | II | ADENOCARCINOMA | STABLE |
| 16377 | 69 | M | CA | 3 | 0 | 0 | II | ADENOCARCINOMA | STABLE |
| 18091 | 83 | M | CA | 3 | 0 | 0 | II | ADENOCARCINOMA | STABLE |
| 18964 | 83 | M | AA | 3 | 0 | 0 | II | ADENOCARCINOMA | STABLE |
| 29112 | 84 | F | CA | 3 | 0 | 0 | II | ADENOCARCINOMA | STABLE |
| 16119 | 68 | F | CA | 3 | 0 | 1 | IV | ADENOCARCINOMA | STABLE |
| 29259 | 67 | F | CA | 2 | 1 | 0 | III | ADENOCARCINOMA | STABLE |
| 30936 | 77 | F | CA | 2 | 1 | 0 | III | ADENOCARCINOMA | UNSTABLE |
| 14276 | 55 | M | AA | 3 | 1 | 0 | III | ADENOCARCINOMA | STABLE |
| 29203 | 65 | F | AA | 3 | 1 | 0 | III | ADENOCARCINOMA | STABLE |
| 30042 | 47 | M | AA | 3 | 1 | 0 | III | ADENOCARCINOMA | STABLE |
| 10863 | 55 | F | AA | 3 | 2 | 0 | III | ADENOCARCINOMA | STABLE |
| 12188 | 46 | F | AA | 3 | 2 | 0 | III | ADENOCARCINOMA | STABLE |
| 16474 | 83 | M | CA | 3 | 2 | 0 | III | ADENOCARCINOMA | STABLE |
| 29145 | 59 | F | CA | 3 | 2 | 0 | III | ADENOCARCINOMA | STABLE |
| 29152 | 56 | M | CA | 3 | 2 | 0 | III | ADENOCARCINOMA | STABLE |
| 22916 | 83 | F | CA | 3 | 1 | X | III | ADENOCARCINOMA | STABLE |
| 11057 | 67 | M | AA | 2 | 1 | 1 | IV | ADENOCARCINOMA | STABLE |
| 29271 | 56 | F | CA | 3 | 1 | 1 | IV | ADENOCARCINOMA | STABLE |
| 3357 | 57 | M | CA | 3 | 2 | 1 | IV | ADENOCARCINOMA | STABLE |
| 16844 | 70 | F | CA | 4 | 1 | 0 | IV | ADENOCARCINOMA | UNSTABLE |
| 29137 | 72 | F | CA | 4 | 2 | 1 | IV | ADENOCARCINOMA | STABLE |

TABLE III
A

| | | | Concentration | Concentration | |
|---|---|---|---|---|---|
| | Genomic | Protein | in Tumor Pool | in Normal Pool | |
| Gene | Position* | Position** | (%) | (%) | SNP ID # |
| KRAS | T41078 > C | R161 > R | 15 | 26 | Novel |
| KRAS | G35393 > A | D173 > D | 99 | 99 | rs4362222 |
| BRAF | A175415 > G | G643 > G | 6 | 9 | rs1042179 |
| CSMD1 | G1501134 > T | L467 > L | 6.9 | 8 | Novel |
| CSMD1 | C1501182 > T | Y483 > Y | 6.6 | 8.5 | rs17066296 |
| CSMD1 | G1586740 > A | Q635 > Q | 75.3 | 61.4 | rs10088378 |
| CSMD1 | G1598562 > A | L848 > L | 41 | 19 | rs3802303 |
| CSMD1 | G1625466 > C | K938 > N | 2.6 | 3.5 | Novel |
| CSMD1 | T1625547 > C | H965 > H | 5 | 2 | Novel |
| CSMD1 | A1627769 > G | T1037 > T | 18.8 | 17 | rs4875703 |
| CSMD1 | G1651453 > A | L1191 > L | 15 | 10 | rs2161752 |
| CSMD1 | C1780223 > G | G1594 > G | 5.1 | 9.1 | Novel |
| CSMD1 | C1804835 > G | L1780 > L | 6 | 2.4 | rs35125470 |
| CSMD1 | C2016045 > T | A2806 > A | 3.3 | 2.5 | Novel |
| CSMD1 | C2031585 > G | L3152 > L | 19.1 | 21.8 | Novel |
| CSMD1 | C2032287 > A | S3192 > S | 24.7 | 20.9 | rs4876056 |
| CSMD1 | C2044547 > T | F3429 > F | 9.9 | 8.4 | rs35043129 |

B

| | Genomic | Protein | Sanger | Tumor(s) with | Tumor |
|---|---|---|---|---|---|
| Gene | Position* | Position** | Results | mutation | Stage |
| KRAS | G5571 > T | G12 > V | Confirmed | 29259, 29112, 16119 | III, II, IV |
| KRAS | G5571 > C | G12 > A | Confirmed | 18964 | II |
| KRAS | G5571 > A | G12 > D | Confirmed | 10236 | II |
| KRAS | G5574 > A | G13 > S | Confirmed | 12304 | II |
| KRAS | TC23577-8 > AA | Q61 > K | Confirmed | 12188 | III |
| KRAS | A23579 > G | Q61 > Q | Confirmed | 30232 | I |
| KRAS | A25207 > G | K117 > R | Confirmed | 11057 | IV |
| BRAF | T130423 > C | V369 > A | Not Detected | | |
| BRAF | A171431 > G | I457 > G | Confirmed | 22916 | III |
| BRAF | C171450 > T | K601 > R | Not Detected | | |
| BRAF | A143126G | S607 > N | Not Detected | | |
| CSMD1-M1 | G1588709 > T | G733 > STOP | Confirmed | 29152 | III |
| CSMD1-M2 | G1595339 > T | G777 > V | Confirmed | 22916 | III |
| CSMD1-M3 | C1687052 > A | P1298 > T | Confirmed | 22916 | III |
| CSMD1-M4 | C1907660 > T | R2476 > STOP | Confirmed | 30042 | III |
| CSMD1-M5 | G1994688 > A | G2682 > S | Confirmed | 10236 | II |
| CSMD1-M6 | C2018150 > G | T2841 > T | Confirmed | 29137 | IV |

| | | |
|---|---|---|
| KRAS | *NC 000012: c25295121-25249447 | **NP 2004976.2 |
| BRAF | *NC 007.12: c140271033-140080751 | **NP 004324.2 |
| CSMD1 | *NC_000008: c4839736-2782789 | **NP_150094 |

What is claimed:

1. A method for assessing risk of node-positive colorectal cancer in an individual, the method comprising detecting mutations in CSMD1 genes in a tumor sample from the individual, the mutations being associated with increased risk of node-positive colorectal cancer.

2. The method of claim 1, wherein the sample is collected during a colonoscopy.

3. The method of claim 1, further comprising administering adjuvant chemotherapy as a result of increased risk of node-positive colorectal cancer.

4. The method claim 1, further comprising administering surgery as a result of the increased risk of node-positive colorectal cancer.

5. The method of claim 1, wherein the sample is a tissue sample.

6. The method of claim 1, wherein the sample is a stool sample.

7. The method of claim 1, further comprising assessing one or more aspects of the individual's personal history.

8. The method of claim 1, wherein one or more aspects are selected from the group comprising sex, race, or age.

9. The method of claim 1, wherein the mutations are detected using an assay.

10. The method of claim 1, wherein the mutations are detected using PCR.

11. The method of claim 1, wherein the mutations are detected using massively parallel picotiter plate pyrosequencing.

12. The method of claim 1, wherein the individual has been previously diagnosed as having node-negative colorectal cancer.

13. The method of claim 1, wherein the individual has been previously diagnosed as having node-positive colorectal cancer.

14. The method of claim 1, wherein the individual has not been previously diagnosed with cancer.

15. A method for assessing risk of node-positive colorectal cancer in an individual, the method comprising detecting mutations in CSMD1 genes in a tumor sample from the individual, the mutations being associated with increased risk of node-positive colorectal cancer, and administering adjuvant chemotherapy as a result of increased risk of node-positive colorectal cancer.

16. A method for assessing risk of node-positive colorectal cancer in an individual, the method comprising detecting mutations in CSMD1 genes in a tumor sample from the individual, the sample being collected during a colonoscopy, the mutations being associated with increased risk of node-positive colorectal cancer, and administering adjuvant chemotherapy as a result of increased risk of node-positive colorectal cancer.

17. A kit for assessing risk of node-positive colorectal cancer in an individual, the kit including reagents capable of assisting in detecting mutations in CSMD1 genes in a sample from the individual, the mutations being associated with increased risk of node-positive colorectal cancer.

18. The kit of claim 17, wherein the sample to be used with the reagents is a stool sample.

19. The kit of claim 17, wherein the sample to be used with the reagents is a tissue sample.

20. The kit of claim 17, wherein the mutations are detected using an assay.
